SEQUENCE LISTING (37 sequences)

SEQ ID NO: 1 - DNA, 2153 bp, Homo sapiens
ccatctcttttggtgcagaaggtgacgggaaacaggccgcagacctgaacttccaaccgtatgtaggcgagaagccggtgccgatactcccactatcccacaatgtcccactgggccccagagtggaagagggcggaggctaatccaagagaccttggggccagctgggatgtcaggggcagcagaggcagtggctggagtggccccttcggccatcagggaccgagagcagcaggctcccgtgaaccaccactctgctttaaaataaagaacaatatggttggtgtggtcattggttacagtggatcaaaaataaaagatctacaacattcgacaaacactaaaatacagatcataaacggggaatctgaagcaaaagtcagaatttttggcaatagggaaatgaaagcaaaggccaaagcggctatagaaacacttattagaaaacaagaaagctacaactcagaatccagtgtggataatgctgcatcccaaacccctattggaagaaatctaggcagaaatgacattgttggagaagctgagccattgtcaaattgggatcgcattagggcagcagtcgtggagtgcgaaaagagaaaatgggcagatctaccaccagttaagaaaaacttttacatagaatccaaagcaacaagctgcatgtctgaaatgcaggtgattaactggagaaaggaaaatttcaacataacgtgtgatgacttgaaaagtggtgaaaagcgtctcattccaaaaccaacttgcaggtttaaagacgcttttcagcaataccctgatcttctgaaaagcataataagggtagggattgtaaagccaacgccaattcagtcacaggcatggccaattattctacaaggaatagatcttatagtagttgcacaaaccggaacagggaaaacattgtcctatctaatgcctgggtttattcatcttgattctcaaccaatatctagagagcaaaggaatgggcctgggatgctagtccttacacccactagagagttggctcttcacgtggaagctgaatgttcaaagtattcatataaaggtctcaaaagcatttgcatatatggtggtagaaacagaaatggacaaatagaagacattagcaaaggtgtagatatcattattgcaactcctgggaggctgaatgacctacaaatgaataactctgtcaacctaagaagcataacctacttggttatagatgaggcagataaaatgctggatatggaatttgaaccccagataaggaagattttattagatgtgcgcccagaccgacagactgttatgacaagtgcaacttggccagatactgtacgtcaactagcactttcttatttgaaagatcctatgattgtttatgttggtaatctgaatctagtggctgtaaatacagtgaagcaaaatataattgttaccacagaaaaagaaaaacgagctctcacccaagaattcgtagagaacatgtcacccaacgacaaagtcatcatgtttgtcagccaaaaacatattgctgatgacttgtcaagcgacttcaatatccaaggcatatctgcagaatcattacatggcaacagtgaacagagtgatcaagagcgagcagtagaggactttaaaagcggaaacataaagatactgattacaactgatatagtatcccgaggtcttgatcttaatgatgtcacacatgtatataattatgatttcccaaggaatattgacgtatatgtacacagagtagggtcattggacggacaggaaagactgcacatcagttccctcatcactcagagagattcgaaaatggccggtgaattgattaaaattctggacagagcaaatcagagtgttccggaagatcttgtagtaatggctgagcaatacaagttaaatcaacaaaagaggcacagagaaacacgatcaagaaaacctggacaaagacgcaaggagttttattttttaagttgaaaagttgtaccaggctactggaagattccaggcatgttaaagatatgcagtattgaatatatgtaaggaagtattggaaacatactagccatttgaagacataactaattcttaaataatactgctaaactttcaaaaaaaaaaaaaaaaaaaaaaaa

SEQ ID NO: 2 - protein, 630 aa, Homo sapiens
MSHWAPEWKRAEANPRDLGASWDVRGSRGSGWSGPFGHQGPRAAGSREPPLCFKIKNNMVGVVIGYSGSKIKDLQHSTNTKIQIINGESEAKVRIFGNREMKAKAKAAIETLIRKQESYNSESSVDNAASQTPIGRNLGRNDIVGEAEPLSNWDRIRAAVVECEKRKWADLPPVKKNFYIESKATSCMSEMQVINWRKENFNITCDDLKSGEKRLIPKPTCRFKDAFQQYPDLLKSIIRVGIVKPTPIQSQAWPIILQGIDLIVVAQTGTGKTLSYLMPGFIHLDSQPISREQRNGPGMLVLTPTRELALHVEAECSKYSYKGLKSICIYGGRNRNGQIEDISKGVDIIIATPGRLNDLQMNNSVNLRSITYLVIDEADKMLDMEFEPQIRKILLDVRPDRQTVMTSATWPDTVRQLALSYLKDPMIVYVGNLNLVAVNTVKQNIIVTTEKEKRALTQEFVENMSPNDKVIMFVSQKHIADDLSSDFNIQGISAESLHGNSEQSDQERAVEDFKSGNIKILITTDIVSRGLDLNDVTHVYNYDFPRNIDVYVHRVGSLDGQERLHISSLITQRDSKMAGELIKILDRANQSVPEDLVVMAEQYKLNQQKRHRETRSRKPGQRRKEFYFLS

SEQ ID NO: 3 - protein, 238 aa, Homo sapiens
PVFAFHHANFPQYVMDVLMDQHFTEPTPIQCQGFPLALSGRDMVGIAQTGSGKTLAYLLPAIVHINHQPYLERGDGPICLVLAPTRELAQQVQQVADDYGKCSRLKSTCIYGGAPKGPQIRDLERGVEICIATPGRLIDFLESGKTNLRRCTYLVLDEADRMLDMGFEPQIRKIVDQIRPDRQTLMWSATWPKEVRQLAEDFLRDYTQINVGNLELSANHNILQIVDVCMESEKDHKL

SEQ ID NO: 4 - protein, 238 aa, Homo sapiens
PVLNFYEANFPANVMDVIARQNFTEPTAIQAQGWPVALSGLDMVGVAQTGSGKTLSYLLPAIVHINHQPFLERGDGPICLVLAPTRELAQQVQQVAAEYCRACRLKSTCIYGGAPKGPQIRDLERGVEICIATPGRLIDFLECGKTNLRRTTYLVLDEADRMLDMGFEPQIRKIVDQIRPDRQTLMWSATWPKEVRQLAEDFLKDYIHINIGALELSANHNILQIVDVCHDVEKDEKL

SEQ ID NO: 5 - protein, 239 aa, Homo sapiens
PTCRFKDAFQQYPDLLKSIIRVGIVKPTPIQSQAWPIILQGIDLIVVAQTGTGKTLSYLMPGFIHLDSQPISREQRNGPGMLVLTPTRELALHVEAECSKYSYKGLKSICIYGGRNRNGQIEDISKGVDIIIATPGRLNDLQMNNSVNLRSITYLVIDEADKMLDMEFEPQIRKILLDVRPDRQTVMTSATWPDTVRQLALSYLKDPMIVYVGNLNLVAVNTVKQNIIVTTEKEKRALT

SEQ ID NO: 6 - protein, 239 aa, Homo sapiens
PTCTFDDAFQCYPEVMENIKKAGFQKPTPIQSQAWPIVLQGIDLIGVAQTGTGKTLCYLMPGFIHLVLQPSLKGQRNRPGMLVLTPTRELALQVEGECCKYSYKGLRSVCVYGGGNRDEQIEELKKGVDIIIATPGRLNDLQMSNFVNLKNITYLVLDEADKMLDMGFEPQIMKILLDVRPDRQTVMTSATWPHSVHRLAQSYLKEPMIVYVGTLDLVAVSSVKQNIIVTTEEEKWSHM

SEQ ID NO: 7 - DNA, 20 bp, Artificial Sequence
  primer_bind (1)..(20)
ttgcttcagattccccgttt

SEQ ID NO: 8 - DNA, 21 bp, Artificial Sequence
  primer_bind (1)..(21)
tttagtgtttgtcgaatgttg

SEQ ID NO: 9 - protein, 4 aa, Artificial Sequence
  DOMAIN (1)..(4)
DEAD

SEQ ID NO: 10 - protein, 13 aa, Artificial Sequence
  MISC_FEATURE (2)..(10): X stands for any amino acid
DXXXXAXXXXGKT

SEQ ID NO: 11 - DNA, 20 bp, Artificial Sequence
  primer_bind (1)..(20)
ggtgccgatactcccactat

SEQ ID NO: 12 - DNA, 20 bp, Artificial Sequence
  primer_bind (1)..(20)
accacagtccatgccatcac

SEQ ID NO: 13 - DNA, 20 bp, Artificial Sequence
  primer_bind (1)..(20)
tccaccaccctgttgctgta

SEQ ID NO: 14 - DNA, 20 bp, Artificial Sequence
  primer_bind (1)..(20)
atcccgaggtcttgatctta

SEQ ID NO: 15 - DNA, 23 bp, Artificial Sequence
  primer_bind (1)..(23)
acttaaaaaataaaactccttgc

SEQ ID NO: 16 - DNA, 20 bp, Artificial Sequence
  primer_bind (1)..(20)
aggttgttgcaggagaccat

SEQ ID NO: 17 - DNA, 20 bp, Artificial Sequence
  primer_bind (1)..(20)
caggtgctgcataactggaa

SEQ ID NO: 18 - protein, 9 aa, Artificial Sequence
  PEPTIDE (1)..(9)
YLMPGFIHL

SEQ ID NO: 19 - protein, 9 aa, Artificial Sequence
  PEPTIDE (1)..(9)
KMAGELIKI

SEQ ID NO: 20 - protein, 9 aa, Artificial Sequence
  PEPTIDE (1)..(9)
ILQGIDLIV

SEQ ID NO: 21 - protein, 9 aa, Artificial Sequence
  PEPTIDE (1)..(9)
IMFVSQKHI

SEQ ID NO: 22 - protein, 9 aa, Artificial Sequence
  PEPTIDE (1)..(9)
ILDRANQSV

SEQ ID NO: 23 - protein, 9 aa, Artificial Sequence
  PEPTIDE (1)..(9)
DLLKSIIRV

SEQ ID NO: 24 - protein, 9 aa, Artificial Sequence
  PEPTIDE (1)..(9)
KILITTDIV

SEQ ID NO: 25 - protein, 9 aa, Artificial Sequence
  PEPTIDE (1)..(9)
LQMNNSVNL

SEQ ID NO: 26 - protein, 9 aa, Artificial Sequence
  PEPTIDE (1)..(9)
VVMAEQYKL

SEQ ID NO: 27 - protein, 9 aa, Artificial Sequence
  PEPTIDE (1)..(9)
LQGIDLIVV

SEQ ID NO: 28 - protein, 9 aa, Artificial Sequence
  PEPTIDE (1)..(9)
VNLRSITYL

SEQ ID NO: 29 - protein, 9 aa, Artificial Sequence
  PEPTIDE (1)..(9)
IILQGIDLI

SEQ ID NO: 30 - protein, 9 aa, Artificial Sequence
  PEPTIDE (1)..(9)
IVYVGNLNL

SEQ ID NO: 31 - protein, 9 aa, Artificial Sequence
  PEPTIDE (1)..(9)
NIDVYVHRV

SEQ ID NO: 32 - protein, 9 aa, Artificial Sequence
  PEPTIDE (1)..(9)
VIDEADKML

SEQ ID NO: 33 - protein, 9 aa, Artificial Sequence
  PEPTIDE (1)..(9)
NLNLVAVNT

SEQ ID NO: 34 - protein, 9 aa, Artificial Sequence
  PEPTIDE (1)..(9)
FIHLDSQPI

SEQ ID NO: 35 - protein, 9 aa, Artificial Sequence
  PEPTIDE (1)..(9)
LNLVAVNTV

SEQ ID NO: 36 - protein, 9 aa, Artificial Sequence
  PEPTIDE (1)..(9)
NLRSITYLV

SEQ ID NO: 37 - protein, 9 aa, Artificial Sequence
  PEPTIDE (1)..(9)
VLTPTRELA

(19) United States
(12) Patent Application Publication
(10) Pub. No.: US 2003/0092083 A1
(43) Pub. Date: May 15, 2003

(54) Cage antigen

(76) Inventors: Doo-il Jeoung, Seoul (KR); Bomsoo Cho, Seoul (KR); Yoon Lim, Seoul (KR); Saeyoung Park, Incheon (KR); Daeyeon Lee, Incheon (KR); Yung-Jue Bang, Seoul (KR); Hankwang Yang, Seoul (KR); Dae-Kee Kim, Seoul (KR)

Correspondence Address: Squire, Sanders & Dempsey L.L.P., 14th Floor, 801 S. Figueroa Street, Los Angeles, CA, 90017-5554, US

(21) Appl. No.: 10/195,117

(22) Filed: Jul. 11, 2002

(30) Foreign Application Priority Data
Aug. 23, 2001 (KR) 10-2001-0050836

(51) Int. Cl. C12Q 1/68; G01N 33/574; C12N 9/64; C12P 21/02; C12N 5/06

(52) U.S. Cl. 435/7.23; 435/226; 435/69.3; 435/320.1; 435/325; 435/6

(57) ABSTRACT

This invention relates to an isolated CAGE gene, which codes for a novel cancer/testis antigen expressed in various cancer cells. Various diagnostic and therapeutic uses arising out of the properties of the DNA and protein are part of this invention.

### BACKGROUND OF THE INVENTION

1. Field of the Invention

The present invention relates to a newly discovered cancer/testis antigen and fragments thereof. The present invention also relates to nucleic acids that encode this antigen. In addition, the present invention relates to methods of screening for persons having cancer by detecting expression of the antigen in such persons. The present invention also relates to CAGE-derived peptides for use as cancer vaccine.

2. Brief Description of the Related Art

There is a growing body of evidence of immune recognition of human cancer. A prerequisite for the successful application of tumor vaccines and other immunological means for the treatment of cancer is the recognition by the immune system of cancer-associated antigens. The immune response requires that T cells recognize and interact with complexes of cell surface molecules, such as HLA (Human Leukocyte Antigens) or MHC (Major Histocompatibility Complex) and certain peptides. The indicated peptides are generally derived from larger molecules, which are processed by the cells that also present the HLA molecules.

The mechanism by which T cells recognize cellular abnormalities has been implicated in cancer. A family of antigens has been found, which are processed into peptides that can lead to lysis of the tumor cells by CTL (Cytotoxic T Lymphocytes) activity. Cancer-associated antigens are those that are either over-expressed or specifically expressed in various types of cancer cells. MAGE-1 was identified in melanoma in 1991. Since then there has been a growing list of cancer-associated antigens with immune stimulatory effect.

Human tumor antigens are recognized by antibodies (Gure et al., Proc. Natl. Acad. Sci., 97: 4198-4203 (2000); Soiffer et al., Proc. Natl. Acad. Sci., 95: 13141-13146 (1998); Gure et al., Cancer Research, 58:1034-1041 (1998); and Scanlan et al., Int. J. Cancer, 76: 652-658 (1998)) or CTLs (Boon et al., J. Exp. Med., 183:725-729 (1996); Wolfel et al., Science, 260: 1281-1284 (1995); Gnjatic et al., J. Immunol., 160, 328-333 (1998); Brandle et al., J. Exp. Med., 183: 2501-2508 (1996); Coulie et al., J. Exp. Med., 180: 35-42 (1994); and Van den Eynde et al., J. Exp. Med., 182:689-698 (1995)). Examples of tumor antigens that are recognized by antibodies include GM2 (Livingston et al., Proc. Natl. Acad. Sci., 84: 2911-2915 (1987)), Her/neu (Disis et al., Cancer Research, 54: 16-20 (1994)), and p53 (Labrecque et al., Cancer Research, 53: 3468-3471 (1993)).

SEREX (Serological identification of antigens by recombinant Expression cloning) methodology has been used to identify such genes. In the SEREX method, autologous serum is used to detect cancer-specific antigens that have immunogenicity. Genes that have been identified by SEREX include those that are over-expressed (Disis et al., Cancer Research, 54: 16-20 (1994)), mutated (Wolfel et al., Science, 260: 1281-1284 (1995); and Robbins et al., J. Exp. Med., 183: 1185-1192 (1996)), alternatively spliced, differentiated (Coulie et al., J. Exp. Med., 180: 35-42 (1994)), and those that are specifically expressed in cancer and normal testis (Chen et al., Proc. Natl. Acad. Sci., 94: 1914-1918 (1997); Chen et al., Proc. Natl. Acad. Sci., 95:6919-6923 (1998); Gure et al., Int. J. Cancer, 72: 965-971 (1997); and Martelange et al., Cancer Res., 60: 3848-3855 (2000)). SEREX has been applied to a variety of tumors, including melanoma, esophageal cancer, renal cancer, astrocytoma, and colon cancer. Many of these SEREX antigens are specifically expressed in cancer and normal testis. There are several C/T (cancer/testis) antigens including MAGE (Gauge et al., J. Exp. Med., 179: 921-930 (1994)), BAGE (Boel et al., Immunity, 2: 167-175 (1995)), and NY-ESO-1 (Jager et al., J. Exp. Med., 187: 265-270 (1998)). These antigens were originally identified in melanoma. It was reported that MAGE and BAGE showed higher expression in metastatic melanoma than in primary melanoma. This indicates that selective expression of C/T antigen is associated with dedifferentiation. Identification of these antigens is necessary for diagnosis and therapeutic development.

Gastric cancer is the major malignancy in South Korea and one of the most common forms of cancer worldwide. Gastric cancer is resistant to chemo- and radiation therapy. Therefore, more effective therapy is needed. Thus far, causative genetic abnormalities associated with gastric cancer have not been found. As a result, it is necessary to establish the basis for immune therapy and to use immunological recognition as a way to gain understanding into the events involved in gastric cancer. Recently, several antigens related to gastric cancer have been identified (Obata et al., Cancer Chemother Pharmacol., 46: S37-42 (2000)). In this invention, we have identified C/T antigens that are specific and indicative of gastric cancer as well as other types of cancer.

### SUMMARY OF THE INVENTION

It is an object of the invention to identify antigens recognized by sera from gastric cancer patients. It is an object of this invention to identify antigens that are specifically expressed in cancerous cells. It is an object of this invention to identify cancer-specific antigens by using SEREX technology. It is an object of this invention to identify C/T antigen by SEREX technology. It is an object of this invention to obtain full-length cDNA sequences of the clone thus identified. It is an object of this invention to isolate mRNA complementary to the above-mentioned gene. It is an object of this invention to localize the above-mentioned gene into the human chromosome. It is still an object of this invention to provide diagnosis for various types of cancer using full or partial cDNA sequences of the above-mentioned gene. It is another object of the invention to identify peptides derived from CAGE antigen which kill tumor cells. It is also an object of the invention to provide a diagnostic assay for various types of cancer using antibodies against CAGE protein. It is still another object of the invention to provide cancer vaccine for the treatment of various types of cancer that express CAGE protein.

The present invention is directed to a purified CAGE protein and fragments thereof that bind HLA-A2 molecule. In particular, the CAGE protein may be about 75 kDa, has DEAD domain, may be endogenously located on the X chromosome, and may be expressed in testis cells and solid tumor cells, but which may not be expressed in leukemia, myeloma or normal cells other than testis cells. The solid tumor may be sarcoma or carcinoma. And the sarcoma or carcinoma may be fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, gastric cancer, hepatic cancer, kidney cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, or retinoblastoma. Preferably, the sarcoma or carcinoma is gastric cancer, cervical cancer, lung cancer, sarcoma, hepatic cancer, kidney cancer, or colon cancer.

The CAGE protein may be as set forth in SEQ ID NO: 2. A CAGE protein that has a sequence homology of about 70%, 80% or 90% to the sequence set forth in SEQ ID NO: 2 is also included.

The present invention is also directed to nucleic acid that encodes the CAGE protein. In particular, the CAGE gene may have the sequence set forth in SEQ ID NO: 1. Nucleic acid that has a sequence homology of about 70%, about 80% or about 90% to the sequence set forth in SEQ ID NO: 1 is also included. The nucleic acid may be cDNA, mRNA or genomic DNA. The invention is also directed to nucleic acid comprising the nucleotides 77-376 and 1,683-1992 of the nucleic acid sequence set forth in SEQ ID NO: 1, and in particular nucleic acid sequence set forth in SEQ ID NO: 7, SEQ ID NO: 11, SEQ ID NO: 14, and SEQ ID NO: 15. The invention is also directed to a vector including expression vector that contains the CAGE gene sequence or fragment thereof, and a promoter that may be inducible or constitutive. The invention is also directed to a host cell that harbors the vector.

The present invention is also directed to a monoclonal or polyclonal antibody that binds specifically to the CAGE protein or a peptide fragment thereof.

The present invention is also directed to a purified CAGE peptide fragment that binds to HLA-A2. In particulare, some of the the purified CAGE peptides may be without limitation YLMPGFIHL (SEQ ID NO: 18), KMAGELIKI (SEQ ID NO: 19), ILQGIDLIV (SEQ ID NO: 20), IMFVSQKHI (SEQ ID NO: 21), ILDRANQSV (SEQ ID NO: 22), DLLKSIIRV (SEQ ID NO: 23), KILITTDIV (SEQ ID NO: 24), LQMNNSVNL (SEQ ID NO: 25), VVMAEQYKL (SEQ ID NO: 26), LQGIDLIVV (SEQ ID NO: 27), VNLRSITYL (SEQ ID NO: 28), IILQGIDLI (SEQ ID NO: 29), IVYVGNLNL (SEQ ID NO: 30), NIDVYVHRV (SEQ ID NO: 31), VIDEADKML (SEQ ID NO: 32), NLNLVAVNT (SEQ ID NO: 33), FIHLDSQPI (SEQ ID NO: 34), LNLVAVNTV (SEQ ID NO: 35), NLRSITYLV (SEQ ID NO: 36), or VLTPTRELA (SEQ ID NO: 37). Further, the purified CAGE peptide may be YLMPGFIHL (SEQ ID NO: 18) or KMAGELIKI (SEQ ID NO: 19).

The present invention is also directed to a method of determining the presence of CAGE gene transcript in a sample, comprising contacting the sample with a probe that hybridizes to a cDNA or mRNA molecule that encodes the CAGE antigen under stringent hybridization conditions, and assaying for the presence of the hybridized cDNA or mRNA molecule. In particular, the presence of the CAGE gene transcript in the sample indicates that the sample contains cancerous cells or cancerous cell extracts, provided that the sample does not contain testes cells or cell extracts. Further, the cells or cell extracts may be from a solid tumor as discussed above.

The invention is also directed to a method of determining the presence of CAGE antigen in a sample, comprising contacting the sample with a ligand that specifically binds to CAGE antigen, and assaying for the presence of the bound ligand-CAGE antigen complex. In particular, the ligand may be a polyclonal or monoclonal antibody. And the detection method may be by Western blot, immunprecipitation, immunofluorescence staining or ELISA.

In another aspect of the invention, the invention is directed to a method of screening for cancer in a subject in which ADP-ribosyltransferase gene GenBank No. XM_{—}0107323; G protein, beta polypeptide2 like gene GenBank No. BC_{—}000672; SOX5 gene GenBank No. NM_{—}006940; ZNF288 gene GenBank No. XM_{—}003095; SOX6 gene GenBank No. AF309034; KNS2 gene GenBank No. XM_{—}007263; HDAC5 gene GenBank No. XM_{—}008359; DDXL gene GenBank No. XM_{—}008972; CAGE gene GenBank No. AY039237; JNK2 gene GenBank No. NM002752; Poly(A) binding protein gene GenBank No. XM018280; or RBPJK/H-2k binding factor gene GenBank No. NM015874 is expressed in the cancerous cell, comprising obtaining a sample from the subject, which is suspected of having cancer, wherein the sample does not contain testes cell, contacting the sample with an antibody that specifically binds to a gene expression product forming an immune complex, wherein detection of the immune complex indicates presence of the cancer in the subject. In particular the detection method may be by but not limited to color reaction, and further by without limitation alkaline phosphatase reaction.

The invention is directed to a method of screening for cancer in a subject in which CAGE antigen gene is expressed in the cancer, comprising obtaining a sample from the subject which is suspected of having cancer, wherein the sample does not contain testes cell, contacting the sample with nucleic acid that specifically hybridizes to a transcript of the gene, and detecting the gene transcript, wherein detection of the gene transcript indicates presence of the cancer in the subject. In particular, the sample may comprise cell lines, tissues, or bodily fluids. And further, the nucleic acid may be as set forth in SEQ ID NO: 7, SEQ ID SEQ ID NO: 11, SEQ ID NO: 14 or SEQ ID NO: 15. And still further, the cancer may be sarcoma or carcinoma as described above.

The invention is also directed to a method of screening for molecules that regulate the expression level of CAGE, comprising obtaining a sample of cancer cells which express CAGE antigen, contacting the sample with antisense oligonucleotides which are complementary nucleic acids to the CAGE gene or aptamers, and determining the expression level of CAGE after the contact, wherein decreased expression level of CAGE in the cancer cells indicates a CAGE expression regulating molecule. In particular, the expression level may be determined by without limitation Northern blot hybridization, RT-PCR, Western blot, immunoprecipitation or immunofluorescence staining.

The invention is also directed to a method of making peptides of CAGE antigen, wherein the CAGE peptide binds to HLA-A2 molecule, comprising making fragments of CAGE antigen and contacting the CAGE peptide with HLA-A2, and assaying for the binding, wherein the peptide that binds to HLA-A2 molecule is isolated. In another aspect of the invention, the invention is direcgted to a method of killing cancer cells comprising contacting cytotoxic T lymphocytes with CAGE peptide that binds to HLA-A2 molecule.

These and other embodiments of the invention will be more fully understood from the following description of the invention, the referenced drawings attached hereto and the claims appended hereto.

### BRIEF DESCRIPTION OF THE DRAWINGS

The present invention will become more fully understood from the detailed description given hereinbelow, and the accompanying drawings which are given by way of illustration only, and thus are not limitative of the present invention, and wherein;

FIGS. 1A-1C show expression analyses of immuno-reactive CAGE gene. (A) RT-PCR was carried out using mRNAs from various normal tissues. Negative control reaction was carried out without template cDNA. Positive control reaction was carried out using cDNA isolated from testis tissue. (B) Expression of CAGE gene in various types of cancer tissue. (C) Expression of CAGE gene in various types of cancer cell lines. CAGE-F and CAGE-R were used as primers for RT-PCR.

FIGS. 2A and 2B show the structure of CAGE gene. (A) The DNA sequence is identified as SEQ ID NO: 1. The amino acid sequence is identified as SEQ ID NO: 2. Primers are underlined. Start and stop codons are denoted as shaded. Various motif sequences of ATP-binding proteins are boxed. CAGE-F and -R are primers that were used for RT-PCR. GSP1 and 2 are primers that were used for RACE (Rapid Amplification of cDNA Ends) reaction. St21.1 and st21.2 are primers that were used for PCR of human×hamster RH clones. (B) Amino acid comparison between various helicase proteins, p72 (SEQ ID NO: 3), p68 (SEQ ID NO: 4), CAGE (SEQ ID NO: 5) and HAGE (SEQ ID NO: 6). Various motif sequences of ATP-binding proteins and S-A-T motif are underlined. Alignment was carried out using GenDoc program.

FIG. 3 shows Northern blot hybridization with a 0.3 Kb insert of CAGE gene cDNA. Each lane was loaded with 2 μg of mRNA from the indicated tissue.

FIG. 4 shows Southern blot hybridization with a 1.9 Kb insert of CAGE cDNA. Each lane was loaded with 10 μg of genomic DNA digested with the indicated restriction enzymes.

FIG. 5 shows localization of CAGE gene on human X chromosome. Fifty nanograms of genomic DNA from each of the 93 radiation hybrid clone (Research Genetics, Inc., Huntsville, Ala., USA) were PCR amplified.

FIGS. 6A-6C show expression, purification, and seroreactivity of CAGE. (A) Expression analysis of CAGE. Western blot using monoclonal anti-His Ab was carried out. E. coli BL21 strain transformed with or without a construct containing full-length CAGE cDNA, which was treated with or without 0.5 mM IPTG. (B) Seroreactivity of sera from gastric cancer patient with the CAGE antigen. Arrow indicates the expressed CAGE protein. Phages without insert were mixed with test clones and served as negative control. Assays were scored positive only when test clones were clearly distinguishable from control phages. Bold arrow indicates test clone. Blank arrow indicates control clone. (C) Purification of CAGE protein was carried out by affinity column chromatography using Ni²⁺-resin. Arrow indicates CAGE protein (75 KDa).

FIGS. 7A and 7B show localization and expression of CAGE protein in C33A cervical cancer cell line. (A) GFP (a, c) or GFP-CAGE (b, d) fusion construct under the control of CMV promoter was transfected into cervical cancer cell line C33A. Localization of GFP protein (a) or GFP-CAGE protein (b) was shown. DAPI images show cell nuclei (c, d). (B) Total cell lysates from stable transfectants of C33A were loaded for SDS-PAGE. Western blot analysis using monoclonal anti-GFP antibody was carried out according to standard procedure. Lanes 1-6 denote stable transfectants of C33A.

FIGS. 8A-8D show CAGE expression levels in (5-aza-2′-deoxycytidine) treated cells. Cancer cell lines (PANC-1 and ACHN), which do not express CAGE, were treated with the indicated concentrations of (5-aza-2′-deoxycytidine) for 4 days (A and B). In addition, cell lines PANC-1 and ACHN were treated with (5-aza-2′-deoxycytidine) (2 μM) for the indicated duration (C and D).

FIGS. 9A and 9B show cell cycle-related expression of CAGE. Cervical cancer cell line was treated with mimosine (400 μM) for 24 h. The culture medium was replaced with fresh medium without mimosine at 0 h and the cells were further cultured for the indicated time period after release from cell cycle block. At each time point, cells were collected for RT-PCR (A), and cell cycle analysis using FACS (fluorescence-activated cell sorting) method (B).

FIG. 10 shows differential expression of CAGE gene in gastric tumors (T) and their corresponding gastric mucosa tissues (N). GAPDH was used as control.

FIGS. 11A-11B show measurement of cytotoxic T cell stimulation by assaying for IFN-γ release. A2-1 and A2-2 peptide-stimulated CD8− T cells were used as effector (indicated as E) and T2 cells (indicated as T or target cells). C plates denote negative control without peptide treatment (B).

### DETAILED DESCRIPTION OF THE INVENTION

As used herein, the term “capable of hybridizing under high stringency conditions” means annealing a strand of DNA complementary to the DNA of interest under highly stringent conditions. Likewise, “capable of hybridizing under low stringency conditions” refers to annealing a strand of DNA complementary to the DNA of interest under low stringency conditions. In the present invention, hybridizing under either high or low stringency conditions would involve hybridizing a nucleic acid sequence (e.g., the complementary sequence to SEQ ID NO: 1 or portion thereof, with a second target nucleic acid sequence). “High stringency conditions” for the annealing process may involve, for example, high temperature and/or low salt content, which disfavor hydrogen-bonding contacts among mismatched base pairs. “Low stringency conditions” would involve lower temperature, and/or lower salt concentration than that of high stringency conditions. Such conditions allow for two DNA strands to anneal if substantial, though not near complete complementarity exists between the two strands, as is the case among DNA strands that code for the same protein but differ in sequence due to the degeneracy of the genetic code. Appropriate stringency conditions which promote DNA hybridization, for example, 6×SSC at about 45° C., followed by a wash of 2×SSC at 50° C. are known to those skilled in the art or can be found in Current Protocols in Molecular Biology, John Wiley & Sons, N.Y. (1989), 6.31-6.3.6. For example, the salt concentration in the wash step can be selected from a low stringency of about 2×SSC at 50° C. to a high stringency of about 0.2×SSC at 50° C. In addition, the temperature in the wash step can be increased from low stringency at room temperature, about 22° C., to high stringency conditions, at about 75° C. Other stringency parameters are described in Maniatis, T., et al., Molecular Cloning: A Laboratory Manual, Cold Spring Harbor Laboratory Press, Cold Spring N.Y., (1982), at pp. 387-389; see also Sambrook J. et al., Molecular Cloning: A Laboratory Manual, Second Edition, Volume 2, Cold Spring Harbor Laboratory Press, Cold Spring, N.Y. at pp. 8.46-8.47 (1989).

As used herein, “C/T” refers to a molecule that is characterized by specific expression in cancer and testis.

As used herein, “CTL” means cytotoxic T lymphocyte.

As used herein, “CAGE” means cancer associated antigen, which is specifically expressed in normal testis and cancer cells.

As used herein, “fragment” refers to a part of a nucleic acid molecule or protein, which retains usable and functional characteristics. In particular, as used with CAGE antigens, peptide fragments have the function of binding to HLA-A2 molecule.

As used herein, “GFP” refers to Green Fluorescent Protein.

As used herein, “GST” refers to Glutathione S Transferase.

As used herein, “His tag” refers to a molecular tag composed of amino acid histidine.

As used herein, “immunohistochemistry” refers to a method that measures level of specific protein in a variety of tissues.

As used herein, “immunoprecipitation” refers to a biological method that quantitatively measures expression level of a protein and also qualitatively the interaction between proteins.

As used herein, “ligand” refers to any molecule or agent, or compound that specifically binds covalently or transiently to a molecule such as a nucleic acid molecule or protein. Ligand may include antibody.

As used herein, “modulates” refers to a change in expression level or biological activity of molecules resulting from specific binding between a molecule and either nucleic acid or protein or small molecule or chemical.

As used herein, “peptide” refers to a molecule that is composed of amino acids. In particular, with respect to CAGE antigen, a peptide fragment has the function of binding to HLA-A2 molecule.

As used herein, “protein” refers to an amino acid sequence, polypeptide, oligopeptide, and polypeptide or portions thereof whether naturally occurring or synthetic.

As used herein, “purified” or “isolated” molecule refers to biological molecules that are removed from their natural environment and are isolated or separated and are free from other components with which they are naturally associated.

As used herein, “RH” or “Radiation Hybrid” refers to a cell line that contains partial complement of chromosomes of human and intact chromosomes of a counterpart such as mouse or hamster.

As used herein, “RT-PCR” refers to a semi-quantitative PCR that uses cDNA as template rather than RNA.

As used herein, “sample” or “biological sample” is referred to in its broadest sense. Any biological sample obtained from an individual, body fluid, cell line, tissue culture, or other source which may contain CAGE polypeptides or polynucleotides of the invention is meant. As indicated, biological samples include body fluids, such as semen, lymph, sera, plasma, urine, synovial fluid, spinal fluid and so on. Methods for obtaining tissue biopsies and body fluids from mammals are well known in the art. Where the biological sample is to include mRNA, a tissue biopsy is the preferred source.

For purposes of the present invention, when mention is made of a sample containing no testis cells in order to detect various types of cancer cells in the sample, this is meant in a functional manner. That is, de minimus amount of testis cells is permitted so long as the presence of the testis cells does not interfere with the assay, the correct reading of the results, and the amount of testis cells in the sample is accounted for. If more than de minimus amount of testis cells is included in the sample, the assay may not give accurate results, but the assay may be permitted so long as the amount of the testis cells is accounted for, the level of CAGE antigen expressed in the testis cells is known and the assay is conducted accordingly.

As used herein, “SEREX” refers to Serological identification of antigens by recombinant Expression cloning.

As used herein, the term “specifically binds” refers to a non-random binding reaction between two molecules, for example between an antibody molecule immunoreacting with an antigen.

CAGE Antigen

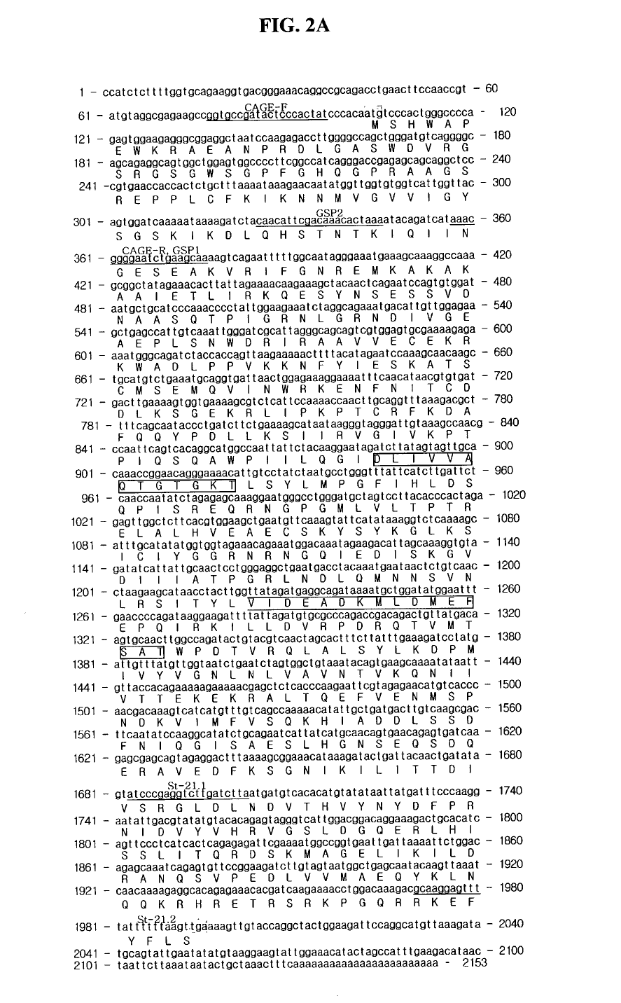

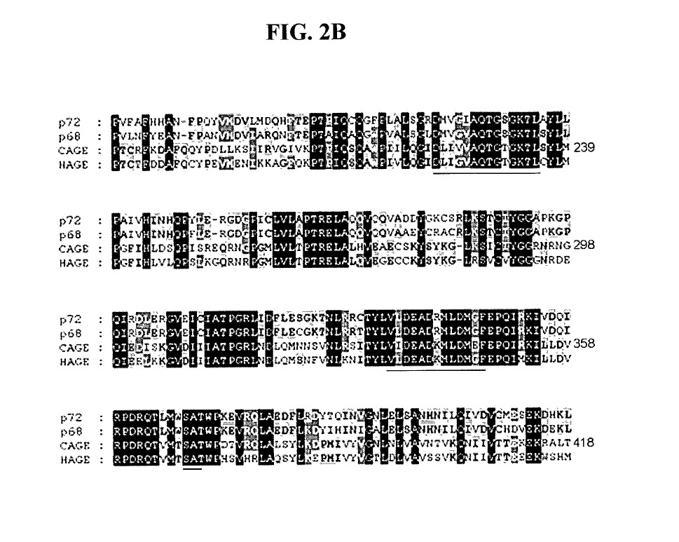

CAGE antigen is an antigen, perhaps a family of antigens, that is expressed in testis and certain cancer cells. In one aspect of the invention, the CAGE antigen gene has been cloned and sequenced and is exemplified in FIG. 2A (SEQ ID NOS: 1 and 2). Furthermore, it has been determined that CAGE possesses sequence properties of an ATP-binding helicase. For example, amino acid sequence at positions 261-273 is the typical A-motif of ATP-binding proteins. Amino acid sequence at positions 374-386 is the typical B-motif of ATP-binding proteins. CAGE also contains a DEAD box domain. The S-A-T motif at 407-409 is conserved in DEAD box proteins. In addition, DEAD box domain contains ATP-dependent helicase activity.

Thus, the CAGE protein contains at least three functional domains: amino acid sequence at positions 301-547 is the helicase (DNA and RNA) domain; amino acid sequence at positions 53-97 is the KH domain; and amino acid sequence at positions 614-631 is the bipartite nuclear localization signal domain. And, CAGE shows homology with RNA helicases p72, p68, and HAGE. However, p72, p68, and HAGE are typically ubiquitously expressed in normal cells, and thus CAGE antigen is distinguished from these RNA helicases.

Thus, with respect to CAGE antigen, it is understood that the CAGE protein is not limited to the one having the specified sequence of SEQ ID NO: 2. In one aspect, the CAGE protein is any protein that is expressed in testes cells and solid tumor cells, but not in other normal cells. Variations in the sequence may be allowed such that about 70% homologous sequence to SEQ ID NO: 2 is permissible. In particular about 75% homology may be allowed. Still more, about 80% homology may be allowed, still further, about 85% homology, and yet further about 90%, more about 95%, and more still about 97% homology or identity may be allowed.

The nucleic acid encoding CAGE protein may also include variants of SEQ ID NO: 1, and may exhibit homology to SEQ ID NO: 1 in about 70%, about 75%, about 80%, about 85%, about 90%, about 95% or about 97% identity. In one aspect of the invention, the variants exhibit DEAD domains, ATP-dependent helicase activity and so on a disclosed above.

Homology

Alignment of amino acid or nucleic acid sequences to determine homology is preferably determined by using a “sequence comparison algorithm.” Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

An example of an algorithm that is suitable for determining sequence similarity is the BLAST algorithm, which is described in Altschul, et al., J. Mol. Biol. 215:403-410 (1990). Software for performing BLAST analyses is publicly available through the National Center for Biotechnology Information (http:H/www.ncbi.nlm.nih.gov/). The BLAST algorithm performs a statistical analysis of the similarity between two sequences (see, e.g., Karlin & Altschul, Proc. Natl Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance.

Isolation of C/T Antigen

Initially, we screened 5×10⁵ recombinant clones from cDNA expression library (primary library) made from human testis mRNA or SNU601 mRNA or MKN74 mRNA. Pooled sera from four gastric cancer patients were used for screening each cDNA expression library. Excluding the false positive clones encoding immunoglobulin fragments, we identified 39 independent immune-reactive clones. These clones were purified, excised, and converted to plasmid form. Their inserts were sequenced. Table 1 shows a list of twelve genes identified in this screen.

TABLE 1
Genes isolated by SEREX of cDNA expression libraries
of human testis and gastric cancer cell lines

| Designa- | | | SEREX | No. |
|---|---|---|---|---|
| tion | Gene | GenBank No. | DB | clones |
| St-1 | ADP-ribosyltransferase | XM_010732 | Yes | 15 |
| St-2 | G protein, beta polypep- | BC_000672 | No | 5 |
| | tide 2 like 1 | | | |
| St-4 | SOX5 | NM_006940 | No | 1 |
| St-8 | ZNF288 | XM_003095 | No | 7 |
| St-9 | SOX6 | AF309034 | No | 1 |
| St-15 | KNS2 | XM_007263 | No | 3 |
| St-17 | HDAC5 | XM_008359 | Yes | 1 |
| St-19 | DDXL | XM_008972 | No | 2 |
| St-21 | Novel | AY039237 | No | 1 |
| | | (in this study) | | |
| St-28 | JNK2 | NM002752 | No | 8 |
| St-30 | Poly(A) binding protein | XM018280 | No | 35 |
| St-31 | RBPJK/H-2k binding | NM015874 | Yes | 11 |
| | factor | | | |

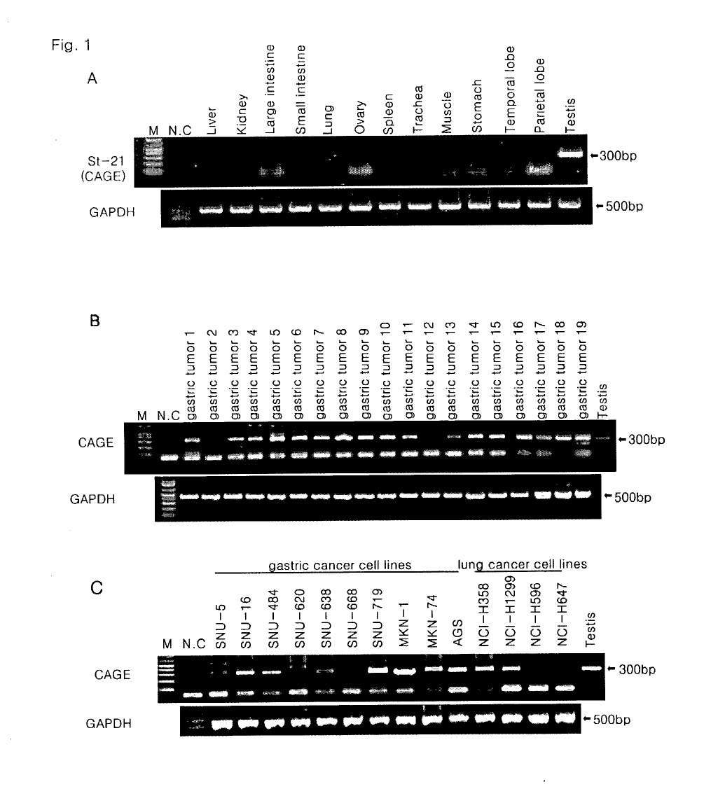

Expression patterns of some of these clones were determined using a panel of normal tissues including liver, kidney, stomach, lung, trachea, large and small intestine, ovary, spleen, muscle, testis, and brain parts (parietal and temporal lobe). We carried out RT-PCR to determine tissue distribution of these clones. As shown in FIG. 1A, clone St-21 was specifically expressed in the testis, and not in any other normal tissue. Coupled with the fact that clone St-21 is also reactive with pooled sera of gastric cancer patients, this clone can be considered to be a C/T antigen.

We further carried out RT-PCR of clone St-21 to determine the expression level of this CAGE gene in a variety of gastric cancer tissues and cell lines. CAGE was expressed in most gastric cancer tissues (FIG. 1B). Furthermore, Table 2 shows universal expression of CAGE in various cancer cell lines such as gastric (7/10), lung (2/4), hepatic (9/10), and cervical carcinoma (6/7). CAGE was highly expressed in gastric cancer tissue (17/19), cervical cancer tissue (20/20), and lung cancer tissue (4/4). Table 2 shows a summary of CAGE expression in various cancer tissues, cancer cell lines, and normal tissues.

TABLE 2
Summary of expression of CAGE

| | | Cancer tissues and | |
|---|---|---|---|
| Normal tissues | | Cancer cell lines | |
| Temporal lobe | 0/2 | Gastric cancer tissue | 17/19 (89%) |
| Parietal lobe | 0/2 | Cervical cancer tissue | 20/20 |
| Spinal cord | 0/1 | Lung cancer tissue | 4/4 |
| Liver | 0/2 | Prostate cancer cell line | 0/2 |
| Kidney | 0/2 | Sarcoma cell line | 1/1 |
| Spleen | 0/3 | Myeloma cell line | 0/4 |
| Stomach | 0/3 | Leukemia cell line | 0/12 |
| Lung | 0/2 | Pancreatic cancer cell line | 0/6 |
| Small intestine | 0/2 | Gastric cancer cell line | 7/10 (70%) |
| Large intestine | 0/2 | Lung cancer cell line | 2/4 (50%) |
| Muscle | 0/2 | Hepatic cancer cell line | 8/10 (80%) |
| Trachea | 0/1 | Cervical cancer cell line | 6/7 (86%) |
| Skin | 0/1 | Melanoma cell line | 0/5 |
| Ovary | 0/2 | Breast cancer cell line | 0/4 |
| Thymus | 0/1 | Kidney cancer cell line | 2/5 (40%) |
| Testis | 2/2 | Colon cancer cell line | 2/4 (50%) |

Widespread distribution in the expression of CAGE in various tumor cells and tumor tissues but not in normal tissues, indicates that the isolated nucleic acid molecule may be used as a diagnostic probe to determine the presence of tumor cells that express CAGE. The fact that CAGE was not expressed in leukemia (0/12) or myeloma cells (0/4) also indicates that expression of CAGE may be specific for solid tumors.

The CAGE gene was completely sequenced (FIG. 2A). We performed 5′-RACE for sequencing of the 5′ ends of CAGE. We discovered that this clone contains DEAD box domain. Amino acid sequence at positions 261-273 is the typical A-motif of ATP-binding proteins. Amino acid sequence at positions 374-386 is the typical B-motif of ATP-binding proteins. The S-A-T motif at 407-409 is conserved in DEAD box proteins. Proteins having DEAD box are known to play a role in RNA metabolism, spermatosis, embryosis, and cell growth (Linder et al., Nature, 337: 121-122 (1989)). ATP-dependent helicases typically contain DEAD box domain (Hirling et al., Nature, 339: 562-564 (1989); and Iggo et al., EMBO J., 8: 1827-1831 (1989)). CAGE contains three functional domains. Amino acid sequence at positions 301-547 is the helicase (DNA and RNA) domain. Amino acid sequence at positions 53-97 is the KH domain. Amino acid sequence at positions 614-631 is the bipartite nuclear localization signal domain. CAGE also shows homology with RNA helicases p72, p68, and HAGE (FIG. 2B). Recently, two antigens, SAGE and HAGE, were found to contain the DEAD box domain. We did not find any mutation associated with the CAGE gene (data not shown).

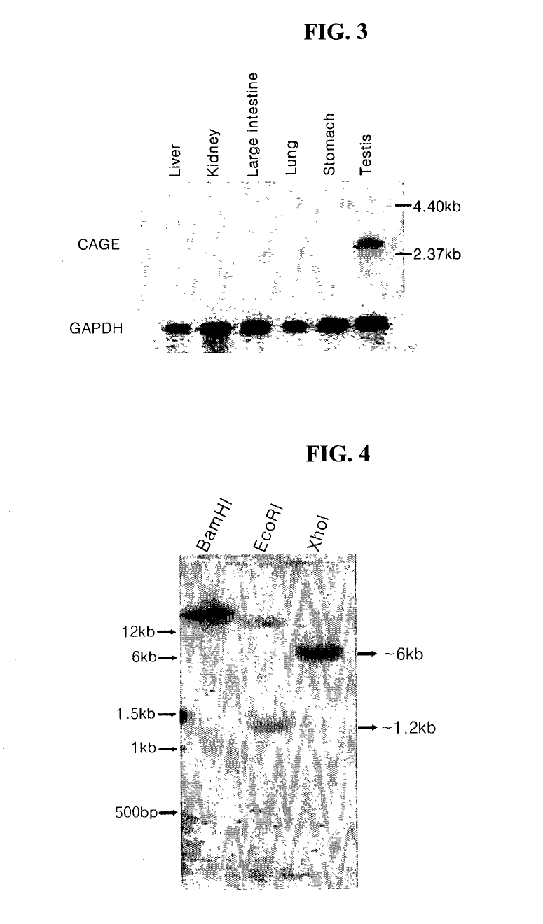

We carried out Northern blot hybridization, and found a single 2.3 Kb transcript (FIG. 3). This indicates that we indeed cloned the full-length cDNA sequence of CAGE or at least a gene in the family of CAGE genes. Further, we used a 0.3 Kb fragment of the CAGE cDNA as probe. We carried out Southern blot hybridization to confirm that the CAGE gene exists as a single copy. Genomic DNA from gastric cancer cell line AGS was digested with the indicated restriction enzymes and Southern blot hybridized using the 1.9 Kb cDNA of CAGE gene as probe. As shown in FIG. 4, it produced a single band indicating that CAGE exists as a single copy. This is unusual because many C/T antigens, including MAGE, exist in multiple copies.

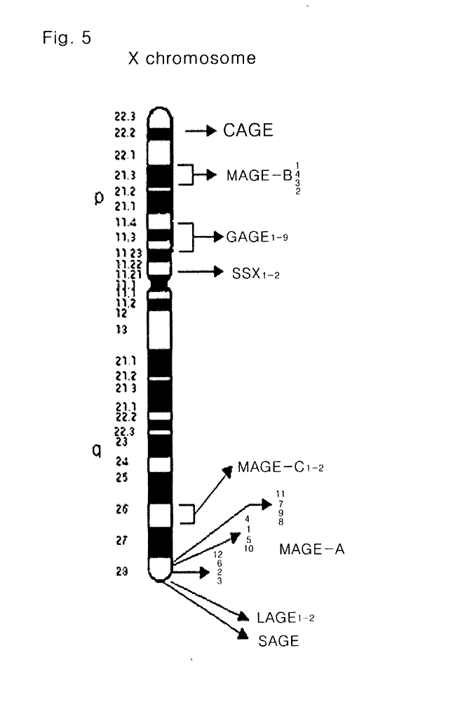

We carried out PCR using human x hamster RH panel to localize CAGE gene to human chromosome. We found that CAGE gene was localized to chromosome Xp22 based on genomic PCR of human x hamster RH panel (FIG. 5). Indeed, many C/T antigens such as MAGE, SSX, and LAGE, are located in chromosome X. HAGE is located in chromosome 6.

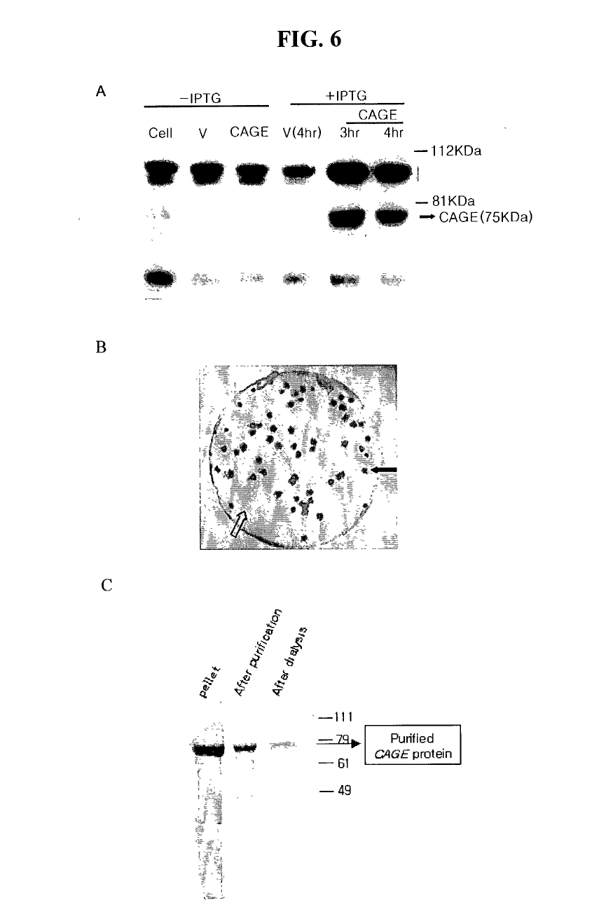

We performed Western blot analysis to determine the size of the CAGE protein. E. coli was transformed with a recombinant vector containing full-length CAGE cDNA. After induction by IPTG the cell lysates were subjected to Western blot analysis using monoclonal anti-His antibody. The size of CAGE protein was determined to be approximately 75 KDa (FIG. 6A). FIG. 6B shows reactivity of the CAGE protein with serum from gastric cancer patients.

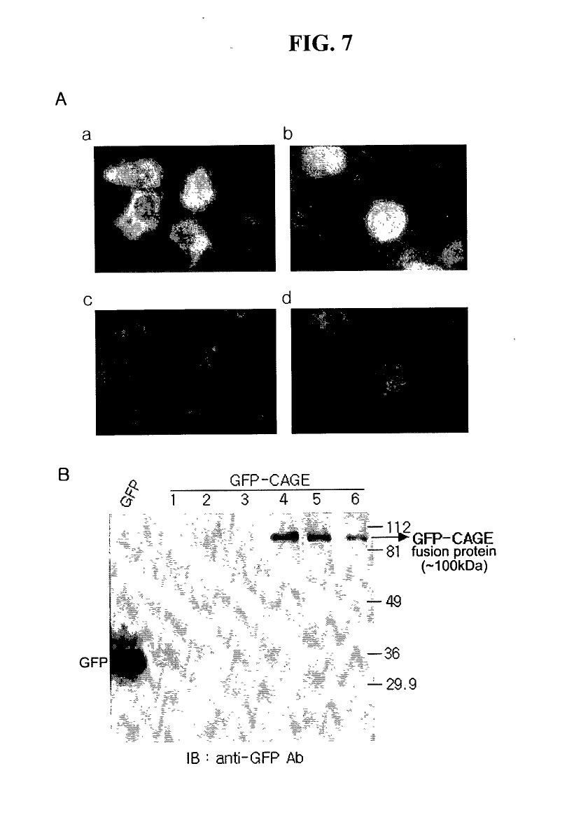

We also purified CAGE protein by using immunoaffinity chromatography using Ni²⁺-resin (FIG. 6C). Using MALDI-TOF sequencing, we confirmed that the band indeed represented CAGE protein. We also transiently transfected GFP-CAGE fusion construct into mammalian cells to localize the CAGE protein. In C33A cells, mostly nuclear localization was shown (FIG. 7A(b)). In other mammalian cells, nuclear, and/or cytoplasmic localization was shown depending on the cell lines transfected.

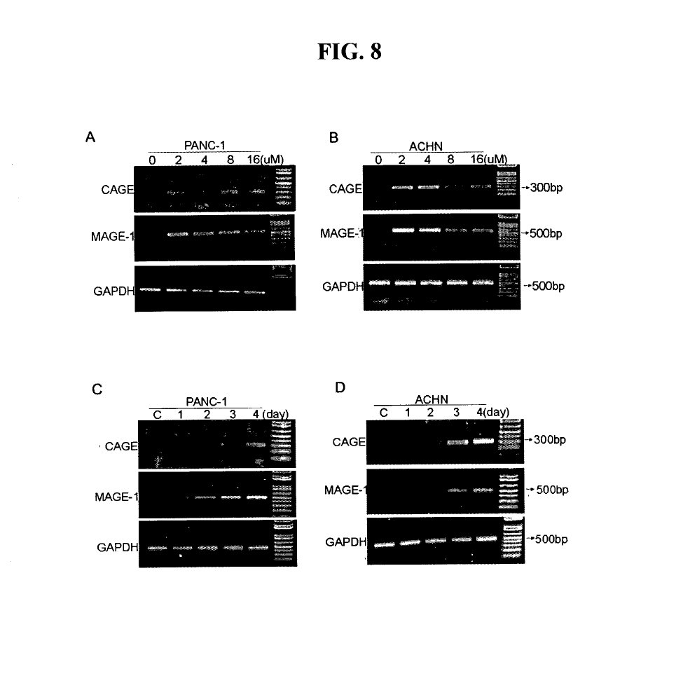

Many of the known C/T antigen genes are methylated in their promoter regions. To determine whether CAGE gene is also methylated, cancer cell lines PANC-1 and ACHN, which do not normally express CAGE gene, were treated with 5-aza-2′-deoxycytidine. As shown in FIGS. 8A-8D, 5-aza-2′-deoxycytidine induced expression of CAGE in dosage and time-dependent manner. This indicates that CAGE gene is methylated in a similar fashion to other C/T antigens, such as MAGE. It is understood that it would be within one of ordinary skill in the art to determine the methylation status of CAGE gene by cloning the promoter sequence of CAGE using any available method such as, but not limited to, genomic inverse PCR.

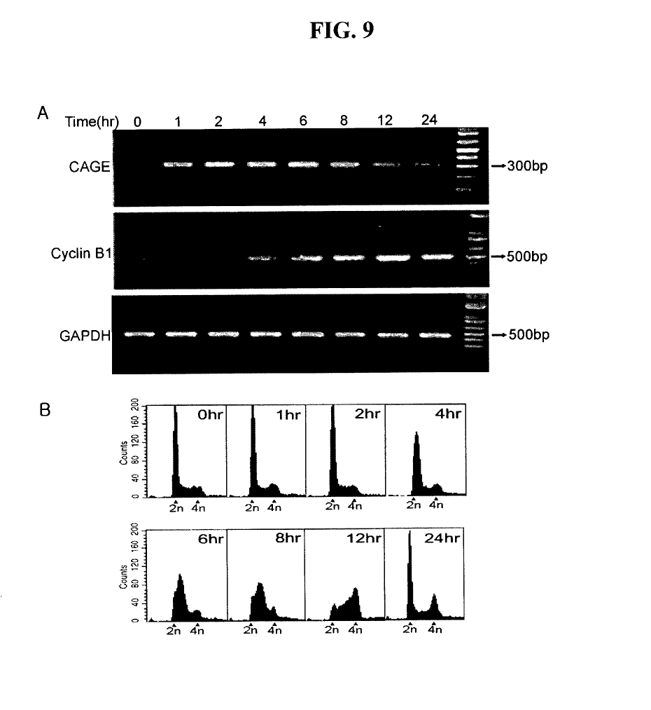

Based on the possibility of CAGE being a cancer-associated gene, we investigated whether the expression of CAGE was under cell cycle control. Mimosine inhibits progression of the cell cycle in late G1 near the G1-to-S phase transition. A cervical cancer cell line was treated with mimosine (400 μM) for 24 h. At each time point after its removal, cell cycle analysis and RT-PCR were carried out. As shown in FIGS. 9A-9B, CAGE was induced as early as 1 hour after mimosine removal. S-phase marker gene cyclin B1 showed maximal induction at 12 hours after release from cell cycle block. Since mimosine blocks cell cycle at G1/S boundary, CAGE is induced at late G1 phase, which conclusion is further supported by the fact that induction of CAGE precedes that of cyclin B1.

We next assayed for CAGE overexpression in certain types of cancer compared with surrounding normal tissue. Sixteen pairs of gastric tumor tissue and their corresponding gastric mucosal tissue were used. These gastric mucosa tissues appear to be homogeneous unlike gastric tumor tissues. cDNA microarray analyses using 2,400 human genes to check the homogeneity of gastric mucosa tissue indicated that there was no distinct pattern of expression among these tissues (data not shown).

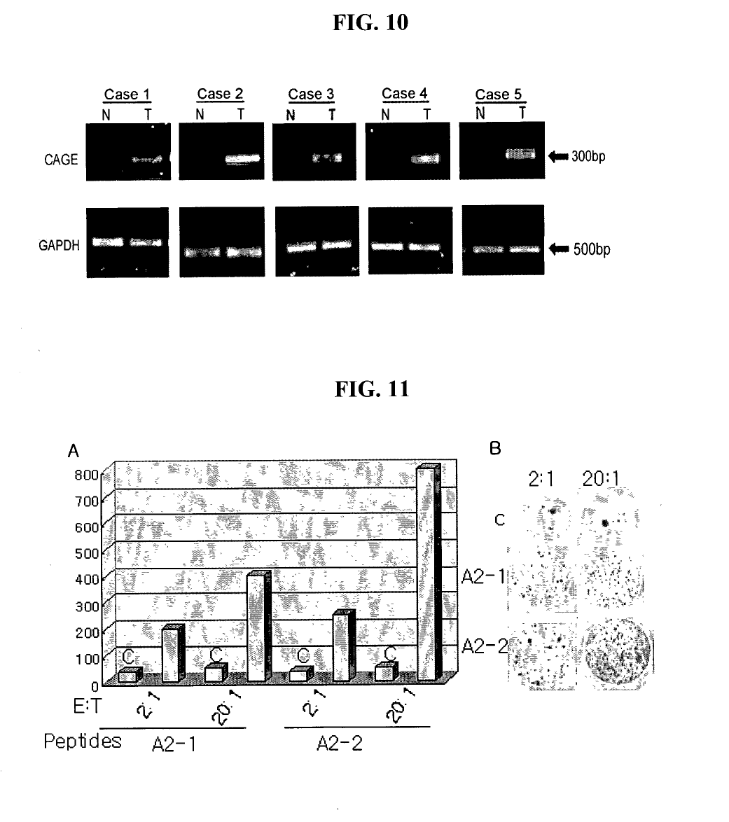

We found that CAGE was overexpressed in over 50% (9/16) of gastric tumor tissues compared with their surrounding gastric mucosal tissues (FIG. 10). Most of these gastric tumor tissues (6/9) that showed overexpression, had a phenotype of being poorly differentiated. These results indicate that not only is CAGE expression associated with gastric tumorigenesis, but that it is also associated with the phenotype of the gastric tissue being poorly differentiated. CAGE gene expression appears to be restricted to tumors and germ-line cells, and thus potentially codes for tumor specific antigen recognized by T lymphocytes. It is noted that spermatogenic cells that express CAGE do not express HLA molecules that would present CAGE antigen to T cells.

The identification of the novel C/T antigen of the invention leads to enhanced possibilities for therapeutic cancer vaccination with tumor-specific antigenic peptides. Peptides of some C/T antigens have been shown to induce CD8+ T cells. The induction of CD8+ T cells often leads to lysis of tumor cells. In this invention, we determined that CAGE protein and peptide fragments thereof may be useful as cancer vaccine. First, based on protein database search following the Parker et al. scheme that ranks potential 8-mer, 9-mer, or 10-mer peptides based on predicted half-time of dissociation to HLA class I molecules and the deduced coefficient tables, we identified peptide fragments of CAGE protein that have a high probability of binding to HLA molecules. The peptide fragment may be any length so long as the fragment binds to HLA-A2 so that the peptide is properly presented to CTL to activate CTL (Parker et al., “Scheme for ranking potential HLA-A2 binding peptides based on independent binding of individual peptide side-chains”, J. Immunol. 152, 163 (1994)). Preferably, the peptide fragment is at least about 5 amino acids long. Still more preferably, the peptide fragment is about 9 amino acids long. In another aspect, the fragment may be less than about 30 amino acids long.

With these peptides, we stimulated CD8+ T cells isolated from PBL of healthy donors. The activated CD8+ T cells were incubated with target cells (B2). The effect of CD8+ T cells on target cells was measured by ELISPOT assay, which detects the amount of IFN-γ released due to activation of CD8+ T cells.

As shown in FIGS. 11A-11B, two different peptide fragments of CAGE protein induced CD8+ T cells. This indicates that peptide fragments of CAGE protein may be developed as cancer vaccines. It is also understood that CAGE is expressed in a variety of cancer cells and cancer tissues. Therefore, peptide fragments of CAGE protein may be used as vaccines for treatment of various types of cancer, and not necessarily limited to any particular type of cancer that has been exemplified in the instant application, such as gastric cancer, cervical cancer and so on.

CAGE Nucleic Acid

All amino acid sequences of polypeptides encoded by DNA molecules determined herein were predicted by translation of a DNA sequence determined as above. Therefore, as is known in the art for any DNA sequence determined by an automated approach, any nucleotide sequence determined herein may contain some errors. Nucleotide sequences determined by automation are typically at least about 90% identical, more typically at least about 95% to at least about 99.9% identical to the actual nucleotide sequence of the sequenced DNA molecule. The actual sequence can be more precisely determined by other approaches including manual DNA sequencing methods well known in the art.

By “isolated” polynucleotide sequence is intended a nucleic acid molecule, DNA or RNA, which has been removed from its native environment. This includes segments of DNA comprising the CAGE polynucleotides of the present invention isolated from the native chromosome. These fragments include both isolated fragments consisting only of CAGE DNA and fragments comprising heterologous sequences such as vector sequences or other foreign DNA. For example, recombinant DNA molecules contained in a vector are considered isolated for the purposes of the present invention which may be partially or substantially purified.

In addition, isolated nucleic acid molecules of the invention include DNA molecules which comprise a sequence substantially different from those described above but which, due to the degeneracy of the genetic code, still encode CAGE polypeptides and peptides of the present invention. Thus, it would be routine for one skilled in the art to generate the degenerate variants described above, for instance, to optimize codon expression for a particular host.

In another aspect, the invention provides an isolated nucleic acid molecule comprising a polynucleotide which hybridizes under stringent hybridization conditions to a portion of a polynucleotide in a nucleic acid molecule of the invention described above. Hybridizing polynucleotides are useful as diagnostic probes and primers as discussed above. Portions of a polynucleotide which hybridize to the CAGE gene such as set forth in SEQ ID NO: 1, which can be used as probes and primers, may be precisely specified by 5′ and 3′ base positions or by size in nucleotide bases as described above or precisely excluded in the same manner. Preferred hybridizing polynucleotides of the present invention are those that, when labeled and used in a hybridization assay known in the art (e.g. Southern and Northern blot analysis), display the greatest signal strength regardless of other heterologous sequences present in equimolar amounts.

Full-length or partial cDNA sequences of CAGE gene can be used to identify homologous genes under low or high stringency hybridization conditions. One can screen genomic DNA library or cDNA library for identification of CAGE-like genes, and determine expression pattern of these genes by carrying out Northern blot hybridization or RT-PCR.

Variant and Mutant Polynucleotides

The present invention further relates to variants of the nucleic acid molecules which encode portions, analogs or derivatives of CAGE polypeptides, and variant polypeptides thereof including portions, analogs, and derivatives of the CAGE polypeptides. Variants may occur naturally, such as a natural allelic variant. By an “allelic variant” is intended one of several alternate forms of a gene occupying a given locus on a chromosome of an organism. Non-naturally occurring variants may be produced using art-known mutagenesis techniques.

Such nucleic acid variants include those produced by nucleotide substitutions, deletions, or additions. The substitutions, deletions, or additions may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of a CAGE protein of the present invention or portions thereof. Also preferred in this regard are conservative substitutions.

The present application is directed to nucleic acid molecules at least 70%, 75%, 80%, 85%, 90%, 95%, 96%, 97%, 98% or 99% identical to the nucleic acid sequence SEQ ID NO: 1. The above nucleic acid sequences are included irrespective of whether they encode a polypeptide having CAGE activity. This is because even where a particular nucleic acid molecule does not encode a polypeptide having CAGE activity, one of skill in the art would still know how to use the nucleic acid molecule, for instance, as a hybridization probe or primer. Uses of the nucleic acid molecules of the present invention that do not encode a polypeptide having CAGE activity include, inter alia, isolating CAGE gene or allelic variants thereof from a DNA library, and detecting CAGE mRNA expression in biological samples suspected of containing CAGE by Northern Blot analysis or PCR.

The present invention also relates to nucleic acid probes having all or part of a CAGE nucleotide sequence, which are capable of hybridizing under stringent conditions to CAGE nucleic acids. The invention further relates to a method of detecting one or more CAGE nucleic acids in a biological sample obtained from an animal, said one or more nucleic acids encoding CAGE polypeptides, comprising: (a) contacting the sample with one or more of the above-described nucleic acid probes, under conditions such that hybridization occurs, and (b) detecting hybridization of said one or more probes to the CAGE nucleic acid present in the biological sample.

This invention allows for the use of sequences in expression vectors, as well as to transfect host cells and cell lines, be these prokaryotic or eukaryotic cells. This invention also allows for the purification of the protein encoded by CAGE gene. One can insert partial or full-length cDNA sequences of CAGE into an expression vector carrying a suitable promoter. The expression vector may contain various molecular tags for easy purification. Subsequently obtained expression construct may be transformed into any host cell of choice. Cell lysates from the host cell is isolated by established methods well known in the field. The host cell may be prokaryotic or eukaryotic cells.

GFP-containing expression vector may be used to localize CAGE protein in the host cell. An expression vector may be used to stably transfect mammalian cell of choice to determine CAGE gene function. The expression vector may contain inducible or constitutive promoter. Cells that express exogenous CAGE gene may be selected with growth medium containing appropriate concentration of a selective antibiotic or other selective molecules such as G418 or hygromycin.

Variant and Mutant Polypeptides

To improve or alter the characteristics of the CAGE polypeptides of the present invention, protein engineering may be employed. Recombinant DNA technology known to those skilled in the art can be used to create novel mutant proteins or muteins including single or multiple amino acid substitutions, deletions, additions, or fusion proteins. Such modified polypeptides can show, e.g., increased/decreased activity or increased/decreased stability. In addition, they may be purified in higher yields and show better solubility than the corresponding natural polypeptide, at least under certain purification and storage conditions. Further, the polypeptides of the present invention may be produced as multimers including dimers, trimers and tetramers. Multimerization may be facilitated by linkers or recombinantly though heterologous polypeptides such as Fc regions.

Of special interest are substitutions of charged amino acids with other charged or neutral amino acids which may produce proteins with highly desirable improved characteristics, such as less aggregation. Aggregation may not only reduce activity but also be problematic when preparing pharmaceutical formulations, because aggregates can be immunogenic.

The invention provides for isolated CAGE polypeptides comprising the amino acid sequence of full-length CAGE polypeptide having the complete amino acid sequence shown in SEQ ID NO: 2. The polypeptides of the present invention also include polypeptides having an amino acid sequence that is at least 80% identical, more preferably at least 90% identical, and still more preferably 95%, 96%, 97%, 98% or 99% identical to the polypeptide of SEQ ID NO: 2.

Antibodies

Purified full-length CAGE protein can be used to produce monoclonal or polyclonal antibody. Fragments of CAGE protein also can be used to produce monoclonal or polyclonal antibody. Subsequently obtained monoclonal or polyclonal antibody can be used to determine expression of CAGE in various samples including cells, tissues, and body fluids such as but not limited to serum, plasma, and urine. CAGE gene expression in various samples can be assayed by a variety of molecular biological methods, which include but are not limited to in situ hybridization, immunoprecipitation, immunofluorescence staining, Western blot analysis and so on. One can carry out ELISA by using monoclonal antibody against CAGE protein, to determine the amount of CAGE protein in the body fluids of human subjects believed to have an indicated disorder in which CAGE protein is expressed.

One can also use monoclonal or polyclonal antibody made against CAGE protein to identify proteins that interact with CAGE protein. For instance, one can coimmunoprecipitate a CAGE-ligand complex to identify proteins that interact with CAGE protein. In another application of the invention, monoclonal antibody against CAGE protein may be used for measuring the amount of CAGE protein in the sera of cancer patients using well-known assay methods such as ELISA.

Immunoaffinity column chromatography technique may be used to purify CAGE protein. Immunoaffinity column chromatography uses various molecular tags including, but not limited to Flag, His, and GST.

Antibodies of the invention include, but are not limited to, polyclonal, monoclonal, multispecific, human, humanized or chimeric antibodies, single chain antibodies, Fab fragments, F(ab′) fragments, fragments produced by a Fab expression library, anti-idiotypic (anti-Id) antibodies (including, e.g., anti-Id antibodies to antibodies of the invention), and epitope-binding fragments of any of the above. The term “antibody,” as used herein, refers to immunoglobulin molecules and immunologically active portions of immunoglobulin molecules, i.e., molecules that contain an antigen binding site that immunospecifically binds an antigen. The immunoglobulin molecules of the invention can be of any type (e.g., IgG, IgE, IgM, IgD, IgA and IgY), class (e.g., IgG1, IgG2, IgG3, IgG4, IgA1 and IgA2) or subclass of immunoglobulin molecule.

The antibodies of the present invention may be monospecific, bispecific, trispecific or of greater multispecificity. Multispecific antibodies may be specific for different epitopes of a polypeptide of the present invention or may be specific for both a polypeptide of the present invention as well as for a heterologous epitope, such as a heterologous polypeptide or solid support material.

Antibodies of the present invention may be described or specified in terms of the epitope(s) or portion(s) of a polypeptide of the present invention, which they recognize or specifically bind. The epitope(s) or polypeptide portion(s) may be specified as described herein, e.g., by N-terminal and C-terminal positions, by size in contiguous amino acid residues.

Antibodies of the present invention may also be described or specified in terms of their cross-reactivity. Antibodies that do not bind any other analog, ortholog, or homolog of a polypeptide of the present invention are included. Antibodies that bind polypeptides with at least 95%, at least 90%, at least 85%, at least 80%, at least 75%, at least 70%, at least 65%, at least 60%, at least 55%, and at least 50% identity (as calculated using methods known in the art and described herein) to a polypeptide of the present invention are also included in the present invention. Further included in the present invention are antibodies which bind polypeptides encoded by polynucleotides which hybridize to a polynucleotide of the present invention under stringent hybridization conditions (as described herein). Antibodies of the present invention may also be described or specified in terms of their binding affinity to a polypeptide of the invention. Preferred binding affinities include those with a dissociation constant or Kd less than 5×10⁻² M, 10⁻² M, 5 ×10⁻³ M, 10⁻³ M, 5×10⁻⁴ M, 10⁻⁴ M, 5×10⁻⁵ M, 10⁻⁵ M, 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10¹² M, 10⁻¹² M, 5×10⁻¹³ M, 10⁻¹³ M, 5×10⁻¹⁴ M, 10⁻¹⁴ M, 5×10⁻¹⁵ M, or 10⁻¹⁵ M.

The invention also provides antibodies that competitively inhibit binding of an antibody to an epitope of the invention as determined by any method known in the art for determining competitive binding, for example, the immunoassays described herein. In preferred embodiments, the antibody competitively inhibits binding to the epitope by at least 95%, at least 90%, at least 85%, at least 80%, at least 75%, at least 70%, at least 60%, or at least 50%.

Antibodies of the present invention may be used, for example, but not limited to, to purify, detect, and target the polypeptides of the present invention, including both in vitro and in vivo diagnostic and therapeutic methods. For example, the antibodies have use in immunoassays for qualitatively and quantitatively measuring levels of the polypeptides of the present invention in biological samples.

As discussed in more detail below, the antibodies of the present invention may be used either alone or in combination with other compositions. The antibodies may further be recombinantly fused to a heterologous polypeptide at the N- or C-terminus or chemically conjugated (including covalent and non-covalent conjugations) to polypeptides or other compositions. For example, antibodies of the present invention may be recombinantly fused or conjugated to molecules useful as labels in detection assays and effector molecules such as heterologous polypeptides, drugs, radionuclides, or toxins.

The antibodies of the present invention may be generated by any suitable method known in the art. Polyclonal antibodies to an antigen of interest can be produced by various procedures well known in the art. For example, a polypeptide of the invention can be administered to various host animals including, but not limited to, rabbits, mice, rats, etc. to induce the production of sera containing polyclonal antibodies specific for the antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art. The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. In a non-limiting example, mice can be immunized with a polypeptide of the invention or a cell expressing such peptide. Once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Polynucleotides Encoding Antibodies

The invention further provides polynucleotides comprising a nucleotide sequence encoding an antibody of the invention and fragments thereof. The invention also encompasses polynucleotides that hybridize under stringent or lower stringency hybridization conditions, e.g., as defined supra, to polynucleotides that encode an antibody, preferably, that specifically binds to a polypeptide of the invention.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

Assays For Antibody Binding

The antibodies of the invention may be assayed for immunospecific binding by any method known in the art. The immunoassays which can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al, eds, 1994, Current Protocols in Molecular Biology, Vol. 1, John Wiley & Sons, Inc., New York, which is incorporated by reference herein in its entirety). Exemplary immunoassays are described briefly below but are not intended by way of limitation.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., Western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al, eds, 1994, Current Protocols in Molecular Biology, Vol. 1, John Wiley & Sons, Inc., New York at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., 32P or 125I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al, eds, 1994, Current Protocols in Molecular Biology, Vol. 1, John Wiley & Sons, Inc., New York at 10.8.1.

ELISAs comprise preparing antigen, coating the well of a 96 well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al, eds, 1994, Current Protocols in Molecular Biology, Vol. 1, John Wiley & Sons, Inc., New York at 11.2.1.

Diagnostic Assay

The invention also provides diagnostic methods for detecting the expression of the CAGE polynucleotides and polypeptides in a biological sample. One such method involves assaying for the expression of a polynucleotide encoding CAGE polypeptides in a sample from an animal. This expression may be assayed either directly (e.g., by assaying polypeptide levels using antibodies elicited in response to CAGE amino acid sequences or fragments thereof) or indirectly (e.g., by assaying for antibodies having specificity for CAGE amino acid sequences or fragments thereof). The expression of polynucleotides can also be assayed by detecting the CAGE nucleic acids. An example of such a method involves the use of the polymerase chain reaction (PCR) to amplify and detect CAGE nucleic acid sequences in a biological sample.

The present invention also provides a method to diagnose a disorder characterized by the expression of CAGE gene. One can determine expression of CAGE gene by standard methods including Northern blot hybridization, polymerase chain reaction, and Western blot analysis, among other molecular biological techniques that may be used.

The indicated disorder includes tumors of a variety of cancer types, including but not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, gastric cancer, hepatic cancer, kidney cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma and so on.

Where diagnosis of a cancerous state has already been made, the present invention is useful for monitoring progression or regression of the disease state by measuring the amount of CAGE or CAGE expressing cells present in a patient or whereby patients exhibiting enhanced CAGE gene expression will experience a worse clinical outcome relative to patients expressing these gene(s) at a lower level.

Labels

Suitable enzyme labels include, for example, those from the oxidase group, which catalyze the production of hydrogen peroxide by reacting with substrate. Glucose oxidase is particularly preferred as it has good stability and its substrate (glucose) is readily available. Activity of an oxidase label may be assayed by measuring the concentration of hydrogen peroxide formed by the enzyme-labeled antibody/substrate reaction. Besides enzymes, other suitable labels include radioisotopes, such as iodine (¹²⁵I, ¹²¹I), carbon (¹⁴C), sulphur (³⁵S), tritium (³H), indium (¹¹²In), and technetium (^{99m}Tc), and fluorescent labels, such as fluorescein and rhodamine, and biotin.

Further suitable labels for the CAGE polypeptide-specific antibodies of the present invention are provided below. Examples of suitable enzyme labels include malate dehydrogenase, delta-5-steroid isomerase, yeast-alcohol dehydrogenase, alpha-glycerol phosphate dehydrogenase, triose phosphate isomerase, peroxidase, alkaline phosphatase, asparaginase, glucose oxidase, beta-galactosidase, ribonuclease, urease, catalase, glucose-6-phosphate dehydrogenase, glucoamylase, and acetylcholine esterase.

Examples of suitable radioisotopic labels include ³H, ¹¹¹In, ¹²⁵I, ¹³¹I, ³²P, 35S, ¹⁴C, ⁵¹Cr, ⁵⁷To, ⁵⁸Co, ⁵⁹Fe, ⁷⁵Se, ¹⁵²Eu, ⁹⁰Y, ⁶⁷Cu, ²¹⁷Ci, ²¹¹At, ²¹²Pb, ⁴⁷Sc, ¹⁰⁹Pd, etc. ¹¹¹In is preferred isotope where in vivo imaging is used since its avoids the problem of dehalogenation of the ¹²⁵I or ¹³¹I-labeled monoclonal antibody by the liver. In addition, this radionucleotide has a more favorable gamma emission energy for imaging. For example, ¹¹¹In coupled to monoclonal antibodies with 1-(P-isothiocyanatobenzyl)-DPTA has shown little uptake in non-tumors tissues, particularly the liver, and therefore enhances specificity of tumor localization.

Examples of suitable non-radioactive isotopic labels include ¹⁵⁷Gd, ⁵⁵Mn, ¹⁶²Dy, 52^{Tr}, and ⁵⁶Fe.

Examples of suitable fluorescent labels include an ¹⁵²Eu label, a fluorescein label, an isothiocyanate label, a rhodamine label, a phycoerythrin label, a phycocyanin label, an allophycocyanin label, an o-phthaldehyde label, and a fluorescamine label.

Examples of suitable toxin labels include, Pseudomonas toxin, diphtheria toxin, ricin, and cholera toxin.

Examples of chemiluminescent labels include a luminal label, an isoluminal label, an aromatic acridinium ester label, an imidazole label, an acridinium salt label, an oxalate ester label, a luciferin label, a luciferase label, and an aequorin label.

Examples of nuclear magnetic resonance contrasting agents include heavy metal nuclei such as Gd, Mn, and iron.

Typical techniques for binding the above-described labels to antibodies are provided by Kennedy et al. (1976) Clin. Chim. Acta 70:1-31, and Schurs et al. (1977) Clin. Chim. Acta 81:1-40. Coupling techniques mentioned in the latter are the glutaraldehyde method, the periodate method, the dimaleimide method, the m-maleimidobenzyl-N-hydroxy-succinimide ester method, all of which methods are incorporated by reference herein.

The polypeptides and antibodies of the present invention, including fragments thereof, may be used to detect CAGE expression using biochip and biosensor technology. Bio chip and biosensors of the present invention may comprise the polypeptides of the present invention to detect antibodies, which specifically recognize CAGE. Bio chip and biosensors of the present invention may also comprise antibodies which specifically recognize the polypeptides of the present invention to detect CAGE.

Kit

The invention also includes a kit for analyzing samples for the presence of CAGE in a biological sample. In a general embodiment, the kit includes at least one polynucleotide probe containing a nucleotide sequence that will specifically hybridize with a CAGE nucleic acid molecule, and a suitable container. In a specific embodiment, the kit includes two polynucleotide probes defining an internal region of the CAGE nucleic acid molecule. In a further embodiment, the probes may be useful as primers for polymerase chain reaction amplification.

In another embodiment of the invention, the kit comprises an antibody of the invention, preferably a purified antibody, in one or more containers. In a specific embodiment, the kit of the present invention contains a substantially isolated polypeptide comprising an epitope which is specifically immunoreactive with an antibody included in the kit. Preferably, the kit of the present invention further comprises a control antibody which does not react with the polypeptide of interest. In another specific embodiment, the kit of the present invention contain a means for detecting the binding of an antibody to a polypeptide of interest (e.g., the antibody may be conjugated to a detectable substrate such as a fluorescent compound, an enzymatic substrate, a radioactive compound or a luminescent compound, or a second antibody which recognizes the first antibody may be conjugated to a detectable substrate).

Cancer Therapy

T-cell recognition of cellular abnormalities has been implicated to cancer. Many C/T antigens are processed into peptides, which in turn are expressed on cell surfaces, which can lead to lysis of the tumor cells by a specific CTL that recognizes them. For example, a sample of cells, such as blood cells, is contacted with target cells presenting CAGE/HLA complex and capable of provoking CTL to proliferate. The target cells can be a T2 cell (HLA-A2 type) or a transfectant such as COS cell transfected with and expressing a particular HLA and CAGE or cancer cells expressing a particular HLA and CAGE with or without transfection.

It is well known that the blood of patients with tumors frequently contains CTL, which recognize complexes of MHC molecules and peptides. One can take a sample of peripheral blood lymphocytes (PBL) from a patient with tumor, and contact the sample with target cells which express certain HLA molecules and CAGE-derived peptides. A proliferation of CTLs, which are specific for that complex, is seen. The proliferation of CTLs can be determined by such methods as IFN-gamma release assay, ⁵¹Cr-release assay and so on. Proliferation of CTLs in the patient's PBL sample indicates that the patient possibly has tumor cells which express that particular CAGE/HLA complex. The activated CTLs are then administered to a subject with the tumor, which is characterized by certain cancer cells presenting complexes of CAGE/HLA molecules. The CTLs then lyse the abnormal cells, thereby achieving the desired therapeutic goal.

Cancer Vaccine

One can also use purified CAGE protein or peptides derived from it as vaccine to stimulate T cells. One can determine CAGE protein sequences for HLA class I-binding peptides on the website (http://www.blmas.dcrt.nih.gov/molbio). These isolated molecules can be combined with adjuvants to produce vaccines for treating disorder characterized by expression of CAGE gene.

It is understood that various databases may be referred to identify peptides of CAGE protein that bind to HLA molecules. Vaccines can be prepared from cells, such as non-proliferative cancer cells, or non-proliferative transfectants, which present CAGE/HLA complexes on their surface. The cells may be transfectants that have been transfected with coding sequences for one or both of the components necessary for providing CTL response, such as CAGE and HLA molecules. The cells may express both HLA and CAGE molecules without transfection. Moreover, the complexes of CAGE and HLA may be used to produce antibodies.

The polypeptide having the amino acid sequence encoded by nucleotide sequence 1-2153 of SEQ ID NO: 1, and polypeptides derived therefrom and peptide fragments also derived therefrom are also part of this invention. These polypeptides alone or in combination with other polypeptides, may be combined with adjuvants to produce vaccines, which would be useful for treating disorders characterized by the expression of CAGE.

In one embodiment, the present invention relates to methods of vaccinating human subjects as a method of cancer therapy or treatment for auto-immune disease. In this way the inventive vaccine can be administered to human patients who are either suffering from, or prone to suffer from cancer or autoimmune disease.

In cancer therapy it is possible to immunize with peptide-carrier combination to induce T cells capable of recognizing and destroying target cancer cells or alternatively immunize to induce antibodies with anti-tumor activity.

The vaccine according to the invention may contain a single peptide according to the invention or a range of peptides which cover different or similar epitopes. In addition or alternatively, a single polypeptide may be provided with multiple epitopes. The latter type of vaccine is referred to as a polyvalent vaccine.

In a preferred embodiment of the invention the peptide is conjugated to a carrier protein such as for example tetanus toxoid, diphtheria toxoid or oxidized KLH in order to stimulate T cell help.

The formation of cancer vaccines is generally known in the art and reference can conveniently be made to Remington's Pharmaceutical Sciences, 17th ed., Mack Publishing Co., Easton, Pa., USA. For example, from about 0.05 ug to about 20 mg per kilogram of body weight per day may be administered. Dosage regime may be adjusted to provide the optimum therapeutic response. For example, several divided doses may be administered daily or the dose may be proportionally reduced as indicated by the exigencies of the therapeutic situation. The active compound may be administered in a convenient manner such as by the oral, intravenous (where water soluble), intramuscular, subcutaneous, intra nasal, intradermal or suppository routes or implanting (eg using slow release molecules by the intraperitoneal route or by using cells e.g. monocytes or dendrite cells sensitised in vitro and adoptively transferred to the recipient). Depending on the route of administration, the peptide may be required to be coated in a material to protect it from the action of enzymes, acids and other natural conditions which may inactivate said ingredients.

For example, the low lipophilicity of the peptides will allow them to be destroyed in the gastrointestinal tract by enzymes capable of cleaving peptide bonds and in the stomach by acid hydrolysis. In order to administer peptides by other than parenteral administration, they will be coated by, or administered with, a material to prevent its inactivation. For example, peptides may be administered in an adjuvant, co-administered with enzyme inhibitors or in liposomes. Adjuvants contemplated herein include resorcinols, non-ionic surfactants such as polyoxyethylene oleyl ether and n-hexadecyl polyethylene ether. Enzyme inhibitors include pancreatic trypsin inhibitor, diisopropylfluorophosphate (DEP) and trasylol. Liposomes include water-in-oil-in-water CGF emulsions as well as conventional liposomes.

The active compounds may also be administered parenterally or intraperitoneally. Dispersions can also be prepared in glycerol liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, chlorobutanol, phenol, sorbic acid, theomersal and the like. In many cases, it will be preferable to include isotonic agents, for example, sugars or sodium chloride. Prolonged absorption of the injectable compositions can be brought about by the use in the composition of agents delaying absorption, for example, aluminium monostearate and gelatin.

Sterile injectable solutions are prepared by incorporating the active compounds in the required amount in the appropriate solvent with various other ingredients enumerated above, as required, followed by filtered sterilization. Generally, dispersions are prepared by incorporating the various sterile active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from a previously sterile-filtered solution thereof.

When the peptides are suitably protected as described above, the active compound may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavoring agent such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound may be incorporated into sustained-release preparations and formulations.

As used herein “pharmaceutically acceptable carrier and/or diluent” includes any and all solvents, dispersion media, coatings antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired.

The principal active ingredient is compounded for convenient and effective administration in effective amounts with a suitable pharmaceutically acceptable carrier in dosage unit form. A unit dosage form can, for example, contain the principal active compound in amounts ranging from 0.5 μg to about 2000 mg. Expressed in proportions, the active compound is generally present in from about 0.5 μg/ml of carrier. In the case of compositions containing supplementary active ingredients, the dosages are determined by reference to the usual dose and manner of administration of the said ingredients.

Delivery Systems

Various delivery systems are known and can be used to administer a compound of the invention, e.g., encapsulation in liposomes, microparticles, microcapsules, recombinant cells capable of expressing the compound, receptor-mediated endocytosis, construction of a nucleic acid as part of a retroviral or other vector, etc. Methods of introduction include but are not limited to intradermal, intramuscular, intraperitoneal, intravenous, subcutaneous, intranasal, epidural, and oral routes. The compounds or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local. In addition, it may be desirable to introduce the pharmaceutical compounds or compositions of the invention into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

In a specific embodiment, it may be desirable to administer the pharmaceutical compounds or compositions of the invention locally to the area in need of treatment; this may be achieved by, for example, and not by way of limitation, local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody or a peptide of the invention, care must be taken to use materials to which the protein does not absorb. In another embodiment, the compound or composition can be delivered in a vesicle, in particular a liposome. In yet another embodiment, the compound or composition can be delivered in a controlled release system. In one embodiment, a pump may be used. In another embodiment, polymeric materials can be used. In yet another embodiment, a controlled release system can be placed in proximity of the therapeutic target, i.e., the brain, thus requiring only a fraction of the systemic dose.

A composition is said to be “pharmacologically or physiologically acceptable” if its administration can be tolerated by a recipient animal and is otherwise suitable for administration to that animal. Such an agent is said to be administered in a “therapeutically effective amount” if the amount administered is physiologically significant. An agent is physiologically significant if its presence results in a detectable change in the physiology of a recipient patient.

Other Uses for CAGE

This invention also provides a method of identifying aptamers using standard procedures well known in the art. Aptamers are nucleic acid molecules that bind to protein of interest. Thusly obtained aptamers can be used as an alternative to antibody.

This invention allows for antisense nucleic acid molecules directed to inhibiting CAGE or related gene expression. Thus obtained antisense nucleotide molecules may be transfected into mammalian cells of choice and expression profile analysis may be obtained in response to antisense nucleic acid treatment, which reveals genes that are closely related to the function of the CAGE gene. For example, one can transfect antisense nucleic acids and determine the gene expression profile by cDNA microarray analysis to identify genes modulated by antisense nucleotides.

In particular, one can carry out yeast two-hybrid analysis to identify genes that interact with CAGE gene. Yeast two-hybrid analysis is carried out according to standard procedures well known in the field. Full-length or partial cDNA sequences of CAGE gene is used as bait to identify genes that interact with CAGE. In another aspect of the invention, CAGE gene is randomly mutated in situ. This way, domains associated with specific functions of CAGE are identified. In another aspect, mutated CAGE genes are transfected into prokaryotic cells such as E. coli or mammalian cells to produce mutant proteins. Thus produced mutant proteins are biochemically compared with wild type CAGE.

In another aspect of the invention, the invention allows for the detection of autoantibodies against CAGE protein in the sera of patients with cancer. In particular, SEREX is applied to determine the presence of autoantibodies against CAGE protein. For example, λ-ZAP phages without insert are mixed with test clones (phages with CAGE gene) and co-plated. Plaques are lifted onto nitrocellulose membrane. The membrane containing plaques are incubated with sera of cancer patients or healthy donors. Color reaction reveals the presence of autoantibodies against CAGE protein in the sera of cancer patients. Assay is scored only when test clones are clearly distinguishable from control clones.

In yet another aspect of the invention, the invention provides for a method of measuring the concentration of autoantibodies against CAGE protein in the body fluids of cancer patients. Either full-length or truncated forms of CAGE protein may be used. The amount of autoantibodies against CAGE protein may be typically measured by ELISA or Western blot analyses. For the ELISA method peptide fragments of CAGE protein may be used as well as the full-length and other truncated versions.

The following examples are offered by way of illustration of the present invention, and not by way of limitation.

### EXAMPLES

### Example 1

### Construction of cDNA Expression Library

A total of 5 μg of human testicular mRNA (Clontech Company, Palo Alto, Calif., USA) or mRNA of SNU601 or MKN74 gastric cancer cell line was used for the construction of cDNA expression library. Construction of each cDNA expression library was carried out according to the instruction manual provided by the manufacturer (Stratagene Company, La Jolla, Calif., USA). Briefly, messenger RNA was converted into cDNA by MMLV (Moloney Murine Leukemia Virus) reverse transcriptase. This single stranded cDNA, which contains Xho I restriction site, was converted into double stranded cDNA by DNA polymerase. To this double stranded cDNA, EcoRI adaptor sequence was added by T4 DNA ligase, then cut with XhoI to yield unidirectional cDNA, and subsequently ligated to λ ZAP express vector. Thus ligated cDNA was packaged into phage particles by using Gigapack III Gold packaging extract (Stratagene Company, La Jolla, Calif., USA), which was used to infect XL1-Blue MRF, an E. coli strain. The library consisted on average 2×10⁶ primary recombinants and 5×10⁵ of them were used for immunoscreening.

### Example 2

### Screening of cDNA Expression Library with Pooled Sera from Gastric Cancer Patients

Primary cDNA expression library (human testis cDNA expression library) was screened with pooled sera from four gastric cancer patients. Screening procedure was done according to the instruction manual provided by the manufacturer (Stratagene Company). Briefly, pooled sera from four gastric cancer patients were diluted 1:10 in blocking buffer (KPL), preadsorbed with transfected E. coli lysates, and incubated overnight at room temperature with the nitrocellulose membranes containing the phage plaques (10⁴ plaques/100 mm dish). After washing, followed by incubation with secondary antibody, an anti-human Ig G antibody was conjugated with alkaline phosphatase. The reactive phage plaques were visualized by incubation with NBT (Nitro Blue Tetrazolium, 0.3 mg/ml) and BCIP (5-bromo-4-chloro-3-indolyl-phosphate, 0.15 mg/ml). Immunoreactive clones were tested for reactivity toward diluted sera (1:250) of gastric cancer patients or those of normal healthy individuals by using same screening strategy. Sera from gastric cancer patients were provided by Prof. H. Yang (Seoul National University Hospital, Seoul, South Korea).

### Example 3

### Genes Isolated by SEREX of cDNA Expression Libraries

Immuno-reactive cloned inserts were in vivo excised into the plasmid form according to the instruction manual provided by the manufacturer (Stratagene). Plasmid DNA was purified by commercial kit (Qiagen Company, Westburg, Leusden, the Netherlands). Sequencing reactions were performed by ABI PRISM 310 Genetic Analyzer automated sequencer (Perkin Elmer, Foster City, Calif., USA). Sequence homology searches were performed in the databases provided by the National Center for Biotechnology Information (Bethesda, Md., USA). We identified a total of 39 independent clones that reacted with pooled sera of patients with gastric cancer. These clones did not react with any of 19 sera of healthy donors. 30 out of 39 clones identified in this screen were not previously reported in SEREX database. However, most of these clones showed homology with known genes. Table 1 is a list of twelve genes identified in this screen. The most frequently isolated genes were ADP-ribosyl transferase, RBP JK/H-2k binding factor 2, and poly (A)-binding protein genes, comprising 14, 9, and 33% of the clones, respectively. A combination of RT-PCR and EST database search revealed that most of these clones showed ubiquitous expression pattern.

### Example 4

### Cloning and Sequencing of CAGE Gene

Initially, CAGE gene was sequenced by using universal T3 vector primer sequence (FIG. 2A). We carried out 5′-RACE to determine sequences at the 5′ end of CAGE according to the instruction manual (Life Technologies, Inc., Gaithersburg, Md., USA). Primers GSP1 (5′-TTGCTTCAGATTCCCCGTTT-3′) (SEQ ID NO: 7) and GSP2 (5′-TTTAGTGTTTGTCGAATGTTG-3′) (SEQ ID NO: 8) were used. SEQ ID NO: 1 shows the full cDNA sequence, including start and termination codons, for the CAGE gene. A putative open reading frame is nucleotides 82-1,971 of SEQ ID NO: 1. The boxed areas correspond to motifs typical of the D-E-A-D (SEQ ID NO: 9) -box family of helicases: the DXXXXAXXXXGKT (SEQ ID NO: 10) at amino acid position 261-273 is the A-motif of ATP-binding proteins; the D-E-A-D box at amino acid position 374-386 represents B-motif of ATP binding proteins. The S-A-T motif at position 407-409 is well conserved in all D-E-A-D-box proteins. The sequence data reported in this invention have been deposited with GenBank Database under Accession No. AY039237. We tried to determine genomic structure of CAGE, and found that CAGE lacked introns (data not shown). FIG. 2B shows that CAGE protein exhibits homology with RNA helicases p72 and dp68.

### Example 5

### Expression Analysis of CAGE

Analysis of tissue-specific expression of CAGE was carried out according to standard procedure. All primers were commercially synthesized (Bioneer Company, Chungwon, South Korea). Total RNA from various tissues or cancer cell lines was prepared by using Trizol agent (Life Technologies Inc., Gaithersburg, Md., USA). Gastric cancer cell lines used for determining expression pattern of CAGE were obtained from Korea Cell Line Bank (Seoul, South Korea). Other cancer cell lines used for determining expression pattern of CAGE were obtained from A.T.C.C. Tumor tissues used for determining expression pattern of CAGE were obtained, with informed consent, from cancer patients that underwent surgical resection at Seoul National University Hospital. Total RNA (2 μg) was converted into cDNA by superscript reverse transcriptase (Life Technologies Inc., Gaithersburg, Md., USA). The synthesis of cDNA was carried out according to the manual provided by Life Technologies, Inc. (Gaithersburg, Md., USA). Thus obtained cDNA was used as template for PCR. The primers used in this study (RT-PCR) were as follows: 5′-GGTGCCGATACTCCCACTAT-3′ (sense, SEQ ID NO: 1 1) and 5′-TTGCTTCAGATTCCCCGTTT -3′ (antisense, SEQ ID NO: 7).

RT-PCR reactions consisted of 32 amplification cycles of 30 sec at 94° C., 30 sec at 60° C., and 1 min at 72° C. The reaction yielded a 300-bp PCR product. RT-PCR was carried out in Amp PCR system (Perkin-Elmer, Foster city, Calif., USA). Amplification of GAPDH was performed for 30 cycles with sense primer 5′-ACCACAGTCCATGCCATCAC-3′ (SEQ ID NO: 12) and antisense primer 5′-TCCACCACCCTGTTGCTGTA-3′ (SEQ ID NO: 13). RT-PCR consisted of 30 cycles of 30 sec at 94° C., 30 sec at 60° C., and 1 min at 72° C. After completion of the reaction, PCR products were run on 1.5% agarose, and stained with ethidium bromide.

Total RNAs from various human normal tissues were obtained from Bioneer Company (Chungwon, South Korea). Gastric cancer cell lines, breast cancer cell lines, renal cancer cell lines, and colon cancer cell lines were obtained from Seoul National University Cell Bank (Seoul, South Korea). Hepatoma cell lines, lung cancer cell lines, and cervical carcinoma cell lines were kindly provided by Prof. Y. Bang (Seoul National University Hospital). All cancer cell lines used in this invention were grown in RPMI medium containing 10% FBS. CAGE showed expression in only testis cells among normal tissues (FIG. 1A, Table 2) and showed widespread expression in various cancer tissues and cancer cells (FIGS. 1B, 1C, and Table 2). This widespread expression of CAGE in many cancer cells and tumor tissues but not in normal tissues makes it an ideal target for cancer immunotherapy. CAGE expression was not seen in myeloma (0/4) or leukemic cells (0/12) indicating that its expression is specific for solid tumors.

We carried out experiments to determine whether CAGE was differentially expressed between gastric tumor tissues and their corresponding gastric mucosa tissues (FIG. 10). Gastric tumors and their corresponding gastric mucosa tissues were obtained from gastric cancer patients that underwent surgical resection at Seoul National University Hospital (Seoul, South Korea). Histological grading of gastric tumor tissues was decided according to the WHO classification. The average age of patients was 61 years old. Most of the gastric cancer tissues were of male origin (14/19). Histological grading of these gastric tumor tissues classified phenotypes of them into poor (10/19), signet (3/19), moderate (4/19), and mucinous (2/19). The tumor content of each gastric tumor tissue was well over 70% under microscopic observation. Gastric mucosa tissues were resected further from the site of tumor (>10 cm). For RT-PCR of gastric tumor tissues and their corresponding gastric mucosa tissues, primers 5′-GGTGCCGATACTCCCACTAT-3′ (sense, SEQ ID NO: 11) and 5′-TTGCTTCAGATTCCCCGTTT-3′ (antisense, SEQ ID NO: 7) were used.

Many known C/T antigen genes are methylated. Demethylation induces expression of these C/T antigen genes. One possibility of accounting for aberrant C/T antigen expression in cancer relates to global demethylation. For instance, the promoter region of the MAGE gene contains binding sites for transcriptional activators and this promoter region is methylated in normal somatic cells but demethylated in MAGE-expressing cancer cells and normal testis cells. Another possibility of accounting for aberrant C/T antigen expression is mutations in the regulatory regions in the C/T antigen genes. Extensive sequencing of the promoter region as well as upstream and downstream regulatory regions needs to be done to further shed light on this.

Next, we investigated whether CAGE gene was methylated. A cancer cell line that does not express CAGE was chosen. Cancer cell lines (PANC-1 and ACHN), which do not express CAGE, were treated with various concentrations of 5-aza-2′-deoxycytidine for 4 days (FIGS. 8A and 8B) or with 5-aza-2′-deoxycytidine (2 μM) for various time periods (FIGS. 8C and 8D). 5-aza-2′-deoxycytidine induced CAGE expression in both dose and time-dependent manner. These data indicate that expression of CAGE gene in many of these cancer cell lines is linked with demethylation. For RT-PCR, 5′-GGTGCCGATACTCCCACTAT-3′ (sense, SEQ ID NO: 11) and 5′-TTGCTTCAGATTCCCCGTTT-3′ (antisense, SEQ ID NO: 7) were used. While the mechanism of transcriptional silencing of CAGE in some cancer cell lines may not be clear, it appears that testis-specific expression of CAGE is related at least in part to its demethylation level. In addition, the role of transcription factors in the expression of CAGE gene cannot be ruled out. The absence of transcription factors in other somatic tissues may be responsible for the absence of CAGE expression in these tissues. We also discovered that demethylation activated CAGE in normal cells that do not express it (data not shown).

### Example 6.

To localize CAGE gene on the human chromosome, we used Bridge 4 Radiation hybrid panel (Research Genetics, Inc., Huntsville, Ala., USA). Fifty nanograms of genomic DNA from each of the 93 radiation hybrid clones were PCR amplified with primers that were specific for CAGE. PCR process consisted of 30 cycles of 30 sec at 94° C., 30 sec at 56° C., and 30 sec at 72° C. PCR results were submitted for analysis to the web site of the Whitehead Institute for Biomedical Research. Before carrying out PCR of RH clones, genomic PCR of human and hamster was carried out. Primers used for mapping were sense primer 5′-ATCCCGAGGTCTTGATCTTA-3′ (SEQ ID NO: 14) and antisense primer 5′-ACTTAAAAAATAAAACTCCTTGC-3′ (SEQ ID NO: 15). Chromosomal assignment of CAGE was performed via the web site of the Whitehead Institute for Biomedical Research. As shown in FIG. 5, CAGE gene showed localization into X chromosome.

### Example 7.

To detect the presence of the transcript for CAGE, Northern blot hybridization was carried out. Two μg of poly (A)+ RNA from various normal tissues were separated by 1% formaldehyde agarose gel electrophoresis and blotted onto nylon membrane by capillary transfer method, and fixed to the membrane by UV crosslinking (254 nm, 0.15 J/sq). The membrane was prehybridized at 50° C. for 2 hours in 6×SSC, 5×Denhardt's reagent, 0.5% SDS, 100 μg/ml denatured and fragmented salmon sperm DNA, and subsequently hybridized with a specific 0.3 Kb probe for CAGE. Labeling was performed with random primer DNA labeling kit (Takara Company, OTSU, SHIGA, Japan). Hybridization was performed overnight at 50° C. in solution 6×SSC, 0.5% SDS, 100 μg/ml denatured and fragmented salmon sperm DNA. The membrane was then washed progressively, at first with 2×SSC, 0.1% SDS at room temperature for 15 min and the final wash in 0.5×SSC, 0.1% SDS at 68° C. for 30 min. Autoradiography was conducted at room temperature for three days by using imaging plate (Amersham Pharmacia Biotech Korea, Seoul, South Korea) and analyzed with Typoon Scanner (Amersham Pharmacia Biotech). The same membrane was stripped and hybridized with 0.6 Kb probe for GAPDH. Northern blot hybridization showed single transcript of 2.3 Kb in size (FIG. 3).

### Example 8

### Southern Blot Hybridization

Genomic DNA from gastric cancer cell line AGS was prepared according to standard procedure. Briefly, cells were treated with trypsin and were centrifuged for 3 min at 1,000 rpm. To the pellet, 5 ml of PBS buffer was added and centrifuged for 3 min at 1,000. After centrifugation, 1.2 ml of suspension buffer (10 mM Tris-HCl (pH7.4), 10 mM NaCl, 1.5 mM MgCl₂) was added to cell pellet. After mixing, 8 ml of sucrose/proteinase K buffer (27% sucrose, 1×SSC, 1 mM EDTA, 1% SDS, 20 μg/ml proteinase K) was added. After mixing by gently inverting, incubation was continued overnight at 37° C. This was followed by extraction with 10 ml of phenol/chloroform/isoamyl alcohol (25:24:1). Genomic DNA was spooled out and was precipitated with 1 volume of isopropyl alcohol. DNA was washed with 70% EtOH and was air-dried. Ten μg of genomic DNA from gastric cancer cell line AGS were digested with BamHI, EcoRI, and HindIII. They were separated by agarose gel electrophoresis and were transferred onto nylon membrane by capillary transfer method. 1.9 Kb insert of CAGE cDNA was used as probe. Labeling and hybridization were carried out according to standard procedures well known in the field. Random priming method (Takara Company, Japan) was used for labeling according to the instruction manual provided by the manufacturer. Hybridization was carried out on membrane using 5×SSC, 5×Denhardt×s, 0.5% SDS, and 100 μg/ml denatured salmon sperm DNA, at 68° C. Membrane was then washed progressively, at first with 2×SSC, 0.1% SDS at room temperature for 15 min and the final wash in 0.5×SSC, 0.1×SSC at 68° C. for 30 min. Autoradiography was conducted at room temperature for 3 days by using imaging plate (Amersham Pharmacia Biotech). Southern blot hybridization showed restriction digestion pattern indicating the existence of a single copy of the CAGE gene.

### Example 9

### GFP-CAGE Construct and Transfection

To construct GFP-CAGE fusion, an RT-PCR product of CAGE (1.9 Kb) was subcloned into pEGFP-C1 vector (Clontech). Briefly, the PCR product was cut with KpnI and XhoI (blunt ended) and cloned into KpnI and BamHI (blunt ended) sites of pEGFP-C1 vector. 4 μg of pGFP-CAGE construct under the control of CMV promoter, was transiently transfected into human cervical cancer cell line C33A by lipofectin method. Transient expression of the fusion protein was checked within 48 hours. To visualize expression of the fusion protein, cells were fixed with 3.7% (v/v) formaldehyde, 1:5,000 DAPI (4′, 6-diamidino-2-phenylindole, Cal biochem). As shown in FIG. 7(A)(b), CAGE protein showed mostly nuclear localization in the C33A cells.

For stable transfection, 4 μg of pEGFP-CAGE fusion construct was transfected along with lipofectin. Transfection was carried out according to the instruction manual provided by the manufacturers (Clontech). C33A cells expressing exogenous pEGFP-CAGE gene were selected in growth medium containing G418 (400 μg/ml). 5 hours after transfection, fresh RPMI medium containing 10% FBS and antibiotics with G418 (400 μg/ml) was added and the selection was carried out until visible colonies appeared (about 15 days). Each colony was picked and cultured for further use. Stable transfectants of GFP and GFP-CAGE were selected by Western blot with mouse monoclonal anti-GFP antibody (Roche Company). To check the expression of CAGE protein, Western blot analysis using monoclonal anti-GFP antibody was carried out according to standard procedure. Briefly, total cell lysates were prepared from C33A cells transfected with pEGFP or pEGFP-CAGE gene. Cell lysates were diluted with sample buffer and boiled for 5 min. Samples were run on 10% SDS-PAGE. After electrophoresis, proteins were transferred onto PVDF membrane at 4° C. for 2 hours at 200 mA. PVDF membrane was incubated with monoclonal anti-GFP antibody (1:20) for 1 hour. Membrane was washed with buffer (1×PBS, 0.2% Tween (v/v)) for a total of 30 min. After washing, membrane was incubated with HRP-conjugated anti-mouse Ab (1:3,000 dilution) for 1 hour. After washing, detection of protein of interest was carried out by using ECL kit according to the manufacturer's protocol (Amersham International, England). As expected, the size of CAGE protein was approximately 75 KDa (FIG. 7B).

### Example 10

### Purification of CAGE Protein and Western Blot Analysis

Cultured cells (E. coli strain BL21) with pET21a vector (Novagen) only and with pET21a-CAGE construct treated with or without 0.5 mM IPTG were lysed in lysis buffer (100 mM NaH₂PO₄, 10 mM Tris-HCl, 8 M urea). Cell lysates were diluted with sample buffer and boiled for 5 min. Samples were run on 10% SDS-PAGE. After electrophoresis, proteins were transferred onto PVDF membrane at 4° C. for 2 hours at 200 mA. PVDF membrane was incubated with blocking buffer for 1 hour. After blocking, the membrane was incubated with monoclonal anti-His Ab (1:20 dilution) for 1 hour. Membrane was washed with buffer (1×PBS, 0.2% Tween 20 (v/v)) for a total of 30 min. After washing, membrane was incubated with HRP-conjugated anti-mouse Ab (1:3,000 dilution) for 1 hour. After washing, detection of protein of choice was carried out by using ECL kit according to the manufacturer's protocol (Amersham International, England). The protein size was determined to be approximately 75 KDa (FIG. 6A).

For purification of CAGE protein, Ni²⁺ resin (Qiagen Company) was used. Purification of CAGE protein by affinity column chromatography was carried out according to standard procedure. Briefly, cultured cells (E. coli strain BL21) with pET-21a-CAGE construct treated with 0.5 mM IPTG were dissolved in lysis buffer (50 mM NaH₂PO₄, 300 mM NaCl, 10 mM imidazole) and sonicated for 5 min. Cell lysates were centrifuged at 1,200 g for 25 min. As CAGE protein pellet is formed (inclusion body), the pellet was dissolved in lysis buffer (100 mM NaH₂PO₄, 10 mM Tris-HCl, 8 M urea, pH8.0). Purification of CAGE protein was carried out according to the instruction manual provided by the manufacturer using Ni-NTA agarose (Qiagen Company, Westburg, Leusden, the Netherlands). Briefly, dissolved pellet was incubated with Ni-NTA agarose (0.5 ml for 200 ml E. coli culture) for 2 hours and the lysate-resin mixture was loaded on to empty column. After washing twice with wash solution (100 mM NaH₂PO₄, 10 mM Tris-HCl, 8 M urea, pH6.7), CAGE protein was eluted with elution solution (100 mM NaH₂PO₄, 10 mM Tris-HCl, 8 M urea, pH5.9). Elution fraction containing CAGE protein was dialysed with PBS buffer. Subsequently obtained purified CAGE protein was injected into mouse for production of monoclonal antibody. The eluted fraction was subjected to MALDI-TOF sequencing to identify the protein, and the band represented CAGE protein (FIG. 6C).

### Example 11

### Cell Cycle Analysis

Cervical cancer cells (C33A) were synchronized by treatment with mimosine (400 μM) for 24 hours. Mimosine inhibits progression of the cell division cycle in late G1 near the G1 -to-S phase transition. At each time point (0, 1, 2, 4, 8, 12, and 24 hours) after mimosine removal, cells were collected for cell cycle analysis and RT-PCR (FIGS. 9A and 9B). For cell cycle analysis, cells were labeled with propidium iodide (50 μg/ml), and DNA was analyzed by FACScan (Becton-Dickinson). For RT-PCR of CAGE, primers CAGE-F and CAGE-R were used. For RT-PCR of cyclin B1, primers 5′-AGGTTGTTGCAGGAGACCAT-3′ (sense, SEQ ID NO: 16) and 5′-CAGGTGCTGCATAACTGGAA-3′ (antisense, SEQ ID NO: 17) were used. PCR was performed for 23 cycles at 95° C. for 30 sec., 60° C. for 30 sec., and 72° C. for 40 sec. FACS was performed according to standard procedure. Briefly, cells were treated with trypsin-EDTA and PBS added. This was followed by centrifugation at 2,500 rpm for 10 min. 200 μl of PBS was added to the pellet. 3 ml of 100% EtOH was added and incubation continued for 10 minutes, then centrifuged at 2,500 rpm for 5 min. The pellet was washed with PBS twice. 0.1% Triton X-100 (1 ml) was added to the pellet, incubated on ice for 5 min, and centrifuged at 2,500 rpm for 5 min. Pellet was washed with PBS twice. To each sample, 200 μl of PBS and 10 μl of RNase A (1 mg/ml) were added. To this, propidium iodide (100 μg/ml) was added and FACScan was followed.

### Example 12

### Identification of Potential HLA Class I-Binding CAGE Peptides

Searching the CAGE protein sequences for HLA class I-binding peptides was performed on the web site: http://blmas.dcrt.nih.gov/molbio (Parker, K. C., M. A. Bednarek, and J. E. Coligan, “Scheme for ranking potential HLA-A2 binding peptides based on independent binding of individual peptide side-chains”, J. Immunol. 152, 163 (1994). Table 3 lists some of the peptides of CAGE protein with high probability of binding to HLA molecules.

TABLE 3
Peptides of CAGE protein having high
probability of binding to HLA-A2 molecules

| | | | Scoring Results |
|---|---|---|---|
| | | | Score (Estimate of |
| | | | Half Time of |
| | | Subsequence | Disassociation of a |
| | Start | Residue | Molecule Containing |
| Rank | Position | Listing | This Subsequence) |
| 1 | 276 | YLMPGFIHL | 690.817 |
| | | (SEQ ID NO:18) | |
| 2 | 576 | KMAGELIKI | 60.655 |
| | | (SEQ ID NO:19) | |
| 3 | 256 | LOG DLIV | 48.478 |
| | | (SEQ ID NO:20) | |
| 4 | 471 | IMEVSQKHI | 47.394 |
| | | (SEQ ID NO:21) | |
| 5 | 584 | ILDRANOSV | 47.295 |
| | | (SEQ ID NO:22) | |
| 6 | 232 | DLLKSIIRV | 44.392 |
| | | (SEQ ID NO:23) | |
| 7 | 519 | KILITTDIV | 24.881 |
| | | (SEQ ID NO:24) | |
| 8 | 359 | LQMNNSVNL | 13.624 |
| | | (SEQ ID NO:25) | |
| 9 | 597 | VVMAEQYKL | 11.757 |
| | | (SEQ ID NO:26) | |
| 10 | 257 | LQGIDLIVV | 11.305 |
| | | (SEQ ID NO:27) | |
| 11 | 365 | VNLHSITYL | 11.096 |
| | | (SEQ ID NO:28) | |
| 12 | 255 | IILQGIDLI | 10.169 |
| | | (SEQ ID NO:29) | |
| 13 | 427 | IVYVGNLNL | 10.169 |
| | | (SEQ ID NO:30) | |
| 14 | 547 | NIDVYVHRV | 8.798 |
| | | (SEQ ID NO:31) | |
| 15 | 374 | VIDEADKML | 8.189 |
| | | (SEQ ID NO:32) | |
| 16 | 432 | NLNLVAVNT | 7.452 |
| | | (SEQ ID NO:33) | |
| 17 | 281 | FIHLDSQPI | 6.599 |
| | | (SEQ ID NO:34) | |
| 18 | 433 | LNLVAVNTV | 6.568 |
| | | (SEQ ID NO:35) | |
| 19 | 366 | NLRSITYLV | 5.286 |
| | | (SEQ ID NO:36) | |
| 20 | 301 | VLTPTRELA | 4.138 |
| | | (SEQ ID NO:37) | |

### Example 13

### Stimulation of CD8+ T Cells by Peptides of CAGE Protein

Major progress in the identification and characterization of human tumor antigens has occurred over the past decade. The development of approaches to analyzing humoral and cellular immune reactivity to cancer in the context of the autologous host has led to the molecular characterization of tumor antigens recognized by CD8+ T cells and antibody. It is well established that peptide epitopes derived from tumor-associated antigens can be recognized by CTLs in the context of MHC molecules. Many of those C/T antigens are recognized by CTL. C/T antigens were first recognized as targets for autologous CTLs in a melanoma patient with an unusual clinical course. Among these antigens, NY-ESO-1 was shown to have the most potent activity in inducing antitumor activity. Peptides of NY-ESO-1 were shown to induce proliferation of CTL based on ELISPOT assay. Over the past decades, a wide range of MHC class I binding peptides derived from tumor cells of mice and humans and recognized by CD8+ T cells have been defined.

IFN-γ is an immunoregulatory cytokine that plays a key role in host defense by exerting anti-proliferative and immunoregulatory activities. IFN-y induces production of cytokines and upregulates the expression of various membrane proteins including class I and II MHC antigens. IFN-γ also influences T-helper cell phenotype determination by inhibiting Th2 differentiation and stabilizing Th1 cells. IFN-γ is produced primarily by activated NK cells, activated Th1 cells, and activated CD8+ T cells. CD8+ T cells that are stimulated by certain peptides release IFN-γ. In this invention, we identified peptides of CAGE protein that induced proliferation of CD8+ T cells.

### Example 14

### Peripheral Blood Lymphocytes (PBL) Isolation

For isolation of PBL, Ficoll-Paque® PLUS (Amersham Pharmacia Biotech) was used. Isolation of PBLs was carried out according to the instruction manual provided by the manufacturer (Amersham Pharmacia Biotech). Briefly, 30 ml of whole blood was mixed with 20 ml of Ficoll-Paque® and centrifuged at 800 g for 20 min. The pellet was transferred to a new tube. 20 ml of PBS buffer was added to the pellet, and centrifuged at 800 g for 10 min. After centrifugation, 10 ml of PBS buffer was added to the pellet. 5 ml of Ficoll-Paque® was added and the contents centrifuged at 800 g for 20 min. 10 ml PBS was added to the pellet for washing, and the contents centrifuged at 800 g for 10 min. Isolated PBLs from healthy donors (HLA-A2 type) were dissolved in RPMI 1640 containing 10% human serum and antibiotics.

### Example 15

### Isolation of CD8+ T Cells from PBLs

For isolation of CD8+ T cells from PBL, CD8 negative isolation kit (DYNAL) was used and isolation was carried out according to the instruction manual provided by the manufacturer. Briefly, 1×10⁷ PBLs dissolved in 200 μl PBS/0.1% BSA, were mixed with heat inactivated 10% FCS and antibody mixture provided and incubated at 2-8° C. for 10 min. After washing the cells with 1 ml PBS/0.1% BSA, the cells were dissolved in 0.9 ml PBS/0.1% BSA and mixed with washed bead and incubated at 20° C. for 15 min. After incubation, using Dynal MPC magnet, non-CD8 T cells were selected from the supernatant containing CD8+ T cells. Thus, non-CD8 T-cells were used as APC (antigen presenting cells) for the CD8+ cytotoxic T lymphocytes.

### Example 16

### IFN-γ ELISPOT Assay for Measuring CD8+ T Cell Stimulation

CD8 depleted PBLs were γ-irradiated (3,000 RAD) and incubated with 2.5 μg/ml β2-microglobulin and 10 μg/ml peptide (for CAGE peptides, CAGE-A2-1; YLMPGFIHL (SEQ ID NO: 18), CAGE-A2-2; KMAGELIKI (SEQ ID NO: 19)) for 1 hour. These cells (1×10⁶ cells/well) were mixed with CD8+ T cells (2.5×10⁵ cells/well) and incubated at 37° C. for 24 hours. IL-2 and IL-7 (Chemicon), 2.5 ng/ml and 10 ng/ml each were added into each wells and incubation was continued for 5 days. After incubation, IFN-γ ELISPOT (Enzyme Linked Immunospot Assay) was carried out according to the instruction manual provided by the manufacturer (DIACLONE). For the IFN-γ capture assay, antibody was coated to PVDF-bottomed-well plates, and blocked with PBS/2% skimmed dry milk. Peptide stimulated or non-stimulated T2 cells (50,000 cells/well) and activated CD8+ T cells (50,000 cells or 100,000 cells) were mixed into RPMI1640 (no serum, no IL-2) and added to the plate. Incubation was carried out in 37° C. CO₂ incubator for 20 hours and ELISPOT assay was carried out according to the instruction manual (R & D systems, Minneapolis, Minn., USA). As shown in FIGS. 11A and 11B, peptides A2-1 and A2-2 derived from CAGE protein showed induction of CD8+ T cells.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the following claims.

What is claimed is:

1. A purified CAGE protein and fragments thereof that bind HLA-A2 molecule.

2. The CAGE protein according to claim 1, which is about 75 kDa, has DEAD domain, is endogenously located on the X chromosome, and is expressed in testis cells and solid tumor cells, but which is not expressed in leukemia, myeloma or normal cells other than testis cells.

3. The CAGE protein according to claim 2, wherein said solid tumor comprises tissues from sarcoma or carcinoma.

4. The CAGE protein according to claim 3, wherein said sarcoma or carcinoma is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, gastric cancer, hepatic cancer, kidney cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, or retinoblastoma.

5. The CAGE protein according to claim 4,wherein said sarcoma or carcinoma is gastric cancer, cervical cancer, lung cancer, sarcoma, hepatic cancer, kidney cancer, or colon cancer.

6. The CAGE protein according to claim 1, which is about 70% homologous to SEQ ID NO: 2.

7. The CAGE protein according to claim 6, which is about 80% homologous to SEQ ID NO: 2.

8. The CAGE protein according to claim 7, which is about 90% homologous to SEQ ID NO: 2.

9. The CAGE protein according to claim 1, having SEQ ID NO: 2.

10. An isolated nucleic acid molecule, which encodes the CAGE protein according to claim 1.

11. The nucleic acid molecule according to claim 10, which is about 70% homologous to SEQ ID NO: 1.

12. The nucleic acid molecule according to claim 11, which is about 80% homologous to SEQ ID NO: 1.

13. The nucleic acid molecule according to claim 12, which is about 90% homologous to SEQ ID NO: 1.

14. The nucleic acid molecule according to claim 10, wherein said nucleic acid molecule is cDNA molecule.

15. The nucleic acid molecule according to claim 10, which is mRNA molecule.

16. The nucleic acid molecule according to claim 10, which is genomic DNA.

17. The nucleic acid molecule according to claim 10, comprising nucleotides set forth in SEQ ID NO: 1.

18. The nucleic acid molecule according to claim 15, comprising nucleotides 77-376 set forth in SEQ ID NO: 1.

19. The nucleic acid molecule according to claim 15, comprising nucleotides 1,683-1,992 set forth in SEQ ID NO: 1.

20. The nucleic acid molecule according to claim 10 comprising SEQ ID NO: 7.

21. The nucleic acid molecule according to claim 10 comprising SEQ ID NO: 11.

22. The nucleic acid molecule according to claim 10 comprising SEQ ID NO: 14.

23. The nucleic acid molecule according to claim 10 comprising SEQ ID NO: 15.

24. A vector comprising the nucleic acid molecule according to claim 10.

25. The vector according to claim 24, wherein said vector is an expression vector comprising the nucleic acid molecule according to claim 10 operably linked to a promoter.

26. The expression vector according to claim 25, wherein the promoter is an inducible promoter.

27. A host cell comprising the vector of claim 24.

28. A purified antibody that binds specifically to the protein according to claim 1.

29. The antibody according to claim 28, wherein said antibody is polyclonal.

30. The antibody according to claim 28, wherein said antibody is monoclonal.

31. A purified CAGE peptide fragment that binds to HLA-A2.

32. The purified CAGE peptide according to claim 31, which is YLMPGFIHL (SEQ ID NO: 18), KMAGELIKI (SEQ ID NO: 19), ILQGIDLIV (SEQ ID NO: 20), IMFVSQKHI (SEQ ID NO: 21), ILDRANQSV (SEQ ID NO: 22), DLLKSIIRV (SEQ ID NO: 23), KILITTDIV (SEQ ID NO: 24), LQMNNSVNL (SEQ ID NO: 25), VVMAEQYKL (SEQ ID NO: 26), LQGIDLIVV (SEQ ID NO: 27), VNLRSITYL (SEQ ID NO: 28), IILQGIDLI (SEQ ID NO: 29), IVYVGNLNL (SEQ ID NO: 30), NIDVYVHRV (SEQ ID NO: 31), VIDEADKML (SEQ ID NO: 32), NLNLVAVNT (SEQ ID NO: 33), FIHLDSQPI (SEQ ID NO: 34), LNLVAVNTV (SEQ ID NO: 35), NLRSITYLV (SEQ ID NO: 36), or VLTPTRELA (SEQ ID NO: 37).

33. The purified CAGE peptide according to claim 32, which is YLMPGFIHL (SEQ ID NO: 18) or KMAGELIKI (SEQ ID NO: 19).

34. A purified antibody which is specific for the CAGE peptide according to claim 31.

35. A method of determining the presence of CAGE gene transcript in a sample, comprising contacting the sample with a probe that hybridizes to a cDNA or mRNA molecule that encodes the CAGE antigen under stringent hybridization conditions, and assaying for the presence of the hybridized cDNA or mRNA molecule.

36. The method according to claim 35, wherein the presence of the CAGE gene transcript in said sample indicates that the sample contains cancerous cells or cancerous cell extracts, provided that the sample does not contain testes cells or cell extracts.

37. The method according to claim 36, wherein said cells or cell extracts are from a solid tumor.

38. The method according to claim 37, wherein said solid tumor comprises sarcoma or carcinoma.

39. The method according to claim 38, wherein said sarcoma or carcinoma is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, gastric cancer, hepatic cancer, kidney cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma.

40. A method of determining the presence of CAGE antigen in a sample, comprising contacting the sample with a ligand that specifically binds to CAGE antigen, and assaying for the presence of the bound ligand-CAGE antigen complex.

41. The method according to claim 40, wherein said ligand is an antibody.

42. The method according to claim 41, wherein said antibody is a monoclonal antibody.

43. The method of claim 40, wherein the presence of CAGE antigen is determined by Western blot, immunprecipitation, immunofluorescence staining or ELISA.

44. A method of screening for cancer in a subject in which ADP-ribosyltransferase gene GenBank No. XM_{—}0107323; G protein, beta polypeptide2 like gene GenBank No. BC_{—}000672; SOX5 gene GenBank No. NM_{—}006940; ZNF288 gene GenBank No. XM_{—}003095; SOX6 gene GenBank No. AF309034; KNS2 gene GenBank No. XM_{—}007263; HDAC5 gene GenBank No. XM_{—}008359; DDXL gene GenBank No. XM_{—}008972; CAGE gene GenBank No. AY039237; JNK2 gene GenBank No. NM002752; Poly(A) binding protein gene GenBank No. XM018280; or RBPJK/H-2k binding factor gene GenBank No. NM015874 is expressed in said cancerous cell, comprising obtaining a sample from said subject who is suspected of having cancer, wherein said sample does not contain testes cell, contacting said sample with an antibody that specifically binds to a gene expression product forming an immune complex, wherein detection of said immune complex indicates presence of said cancer in said subject.

45. The method according to claim 44, wherein said detection is by color reaction.

46. The method according to claim 45, wherein said color reaction is by alkaline phosphatase reaction.

47. A method of screening for cancer in a subject in which CAGE antigen gene is expressed in said cancer, comprising obtaining a sample from said subject who is suspected of having cancer, wherein said sample does not contain testes cell, contacting said sample with nucleic acid that specifically hybridizes to a transcript of said gene, and detecting the gene transcript, wherein detection of said gene transcript indicates presence of said cancer in said subject.

48. The method of claim 47, wherein the sample comprises cell lines, tissues, or bodily fluids.

49. The method according to 47, wherein said nucleic acid is set forth in SEQ ID NO: 7, SEQ ID SEQ ID NO: 11, SEQ ID NO: 14 or SEQ ID NO: 15.

50. The method according to claim 47, wherein said cancer is sarcoma or carcinoma.

51. The method according to claim 50, wherein said sarcoma or carcinoma is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, gastric cancer, hepatic cancer, kidney cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma.

52. A method of screening for molecules that regulate expression level of CAGE, comprising obtaining a sample of cancer cells which express CAGE antigen, contacting said sample with antisense oligonucleotides which are complementary nucleic acids to the CAGE gene or aptamers, and determining the expression level of CAGE after the contact, wherein decreased expression level of CAGE in said cancer cells indicates a CAGE expression regulating molecule.

53. The method of claim 52, wherein the expression level of CAGE is determined by Northern blot hybridization, RT-PCR, Western blot, immunoprecipitation or immunofluorescence staining.

54. A method of making peptides of CAGE antigen, wherein said CAGE peptide binds to HLA-A2 molecule, comprising making fragments of CAGE antigen and contacting said CAGE peptide with HLA-A2, and assaying for the binding, wherein the peptide that binds to HLA-A2 molecule is isolated.

55. A method of killing cancer cells comprising contacting cytotoxic T lymphocytes with CAGE peptide that binds to HLA-A2 molecule.
